(19) United States
(12) Patent Application Publication
(10) Pub. No.: US 2024/0199525 A1
(43) Pub. Date: Jun. 20, 2024

(54) SHP2-Targeting Small Molecules For Use As Anti-Cancer Agents

(71) Applicant: West Virginia University Board of Governors on behalf of West Virginia University, Morgantown, WV (US)

(72) Inventor: Yehenew Agazie, Morgantown, WV (US)

(21) Appl. No.: 18/511,496

(22) Filed: Nov. 16, 2023

Related U.S. Application Data
(60) Provisional application No. 63/384,166, filed on Nov. 17, 2022.

(51) Int. Cl. C07C 65/24; A61P 35/00; C07C 59/64

(52) U.S. Cl. CPC C07C 65/24; A61P 35/00; C07C 59/64

(57) ABSTRACT

The present invention provides a compound of the formula:

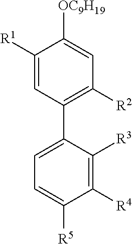

wherein R¹ is a hydrogen or a COOH, R² is a hydrogen or a COOH, R³ is a hydrogen or a COOH, R⁴ is one selected from the group consisting of H, COOH, and CH₂COOH, and R⁵ is one selected from the group consisting of H, COOH, and CH₂COOH. The present invention provides a compound having the formula:

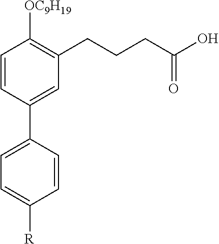

wherein R is COOH or CH₂COOH. Pharmaceutical compositions comprising one of these compounds and at least one of an acceptable pharmaceutical carrier, are provided. Methods of treating a patient having cancer are provided.

### CROSS-REFERENCE TO RELATED APPLICATION

This utility non-provisional patent application claims the benefit of priority to U.S. Provisional Patent Application Ser. No. 63/384,166, filed Nov. 17, 2022. The entire contents of U.S. Provisional Patent Application Ser. No. 63/384,166 are incorporated by reference into this utility non-provisional patent application as if fully rewritten herein.

### STATEMENT REGARDING FEDERALLY FUNDED SPONSORED RESEARCH

This invention was made with government support under grant number R01 CA213996 awarded by the National Institutes of Health. The government has certain rights in the invention.

### BACKGROUND OF THE INVENTION

### 1. Field of the Invention

This invention provides certain compounds and pharmaceutical compositions that are phosphotyrosyl phosphatase 2 (SHP2) targeting molecules for use as anti-cancer agents. The compounds of this invention are inhibitors of SHP2. Methods of treating patients with cancer using the compounds and pharmaceutical compositions of this invention are provided.

### 2. Description of the Background Art

The Src homology 2 containing phosphotyrosyl phosphatase 2 (SHP2) is an oncogenic PTP known to promote tumorigenesis and metastasis in breast cancer and several other cancers particularly in those cancers driven by overexpression of receptor tyrosine kinases (RTKs). As such, there have been concerted efforts to produce specific active site (type I) and allosteric site (type II) inhibitors. Some success has been achieved in developing allosteric inhibitors that went into clinical trials, but there has not been a similar success in developing active site inhibitors that reached a clinical trial. In an effort to fill these gaps, the present invention sets forth certain compounds that are optimized versions of our parent active-site SHP2 inhibitor CNBDA.

### SUMMARY OF THE INVENTION

This invention provides certain compounds that are phosphotyrosyl phosphatase 2 (SHP2) targeting molecules for use as anti-cancer agents. The compounds of this invention are inhibitors of SHP2.

A compound having the formula:

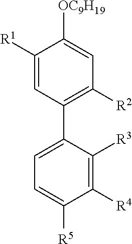

wherein R¹ is a hydrogen or a COOH, R² is a hydrogen or a COOH, R³ is a hydrogen or a COOH, R⁴ is one elected from the group consisting of H, COOH, and CH₂COOH, and R⁵ is one selected from the group consisting of H, COOH, and CH₂COOH, is provided. In certain embodiments of this invention, a compound having this formula is provided wherein R¹ is COOH, and wherein R², R³, and R⁴ are each H, and wherein R⁵ is CH₂COOH (i.e. BPDA2), or wherein R¹, R³, and R⁴ are each H, and wherein R² is COOH, and wherein R⁵ is CH₂COOH (i.e. BPDA6).

The structures of the compounds of this invention are set forth herein and include BPDA1, BPDA2, BPDA3, BPDA4, BPDA5, BPDA6, BPDA7, BPDA8, BPDA9, and BPDA10. Pharmaceutical compositions are provided having the compound of this invention of the formula:

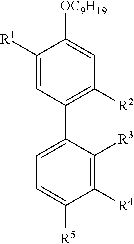

wherein R¹ is a hydrogen or a COOH, R² is a hydrogen or a COOH, R³ is a hydrogen or a COOH, R⁴ is one selected from the group consisting of H, COOH, and CH₂COOH, and R⁵ is one selected from the group consisting of H, COOH, and CH₂COOH, and
at least one acceptable pharmaceutical carrier.

In another embodiment of this invention, a method is provided of treating a patient having cancer comprising administering a therapeutically effective amount of a compound of the formula:

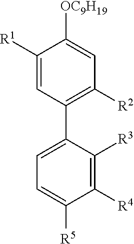

wherein R¹ is a hydrogen or a COOH, R² is a hydrogen or a COOH, R³ is a hydrogen or a COOH, R⁴ is one selected from the group consisting of H, COOH, and CH₂COOH, and R⁵ is one selected from the group consisting of H, COOH, and CH₂COOH.

In another embodiment of this invention, a method is provided of treating a patient having cancer comprising administering a therapeutically effective amount of a pharmaceutical composition comprising a compound having the formula:

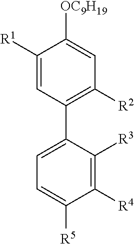

wherein R¹ is a hydrogen or a COOH, R² is a hydrogen or a COOH, R³ is a hydrogen or a COOH, R⁴ is one selected from the group consisting of H, COOH, and CH₂COOH, and R⁵ is one selected from the group consisting of H, COOH, and CH₂COOH, and
an acceptable pharmaceutical carrier. In certain embodiments of this method the compound has the formula wherein R¹ is COOH, and wherein R², R³, and R⁴ are each H, and wherein R⁵ is CH₂COOH, or wherein said compound has the formula wherein R¹, R³, and R⁴ are each H, and wherein R² is COOH, and wherein R⁵ is CH₂COOH.

In another embodiment of this invention, a compound is provided having the formula:

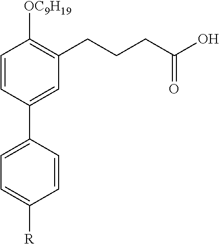

wherein R is COOH or CH₂COOH.

In another embodiment of this invention, a pharmaceutical composition comprising a compound having the formula:

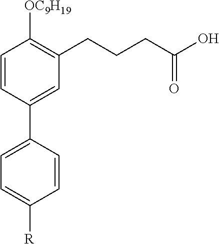

wherein R is COOH or CH₂COOH, and
   at least one pharmaceutically acceptable carrier, is provided

In another embodiment of this invention, a method of treating a patient having cancer is provided comprising administering a therapeutically effective amount of a compound of the formula:

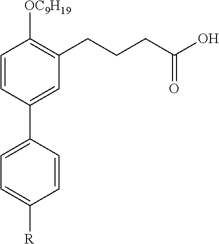

wherein R is COOH or CH₂COOH, for treating said patient.

In another embodiment of this invention, a method of treating a patient having cancer is provided comprising administering a therapeutically effective amount of a pharmaceutical composition comprising a compound having the formula:

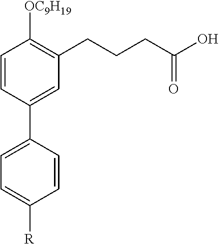

wherein R is COOH or CH₂COOH, and
at least one pharmaceutically acceptable carrier, for treating said patient.

### BRIEF DESCRIPTION OF THE DRAWINGS

FIG. 1 shows the structure of our previous known compound CNBDA.

FIG. 2A shows cellular thermal shift assay (CETSA): MDA-MB468 lysates treated with BPDA2.

FIG. 2B shows cellular thermal shift assay (CETSA): MDA-MB468 cell lysates not treated with BPDA2.

FIG. 2C shows cellular thermal shift assay (CETSA): JIMT-1 cell lysates treated with BPDA2.

FIG. 2D shows cellular thermal shift assay (CETSA): JIMT-1 cell lysates not treated with BPDA2.

FIG. 2E shows cellular thermal shift assay (CETSA): Band density bar graph comparing stability of the target SHP2 and the non-target EGFR in the MDA-MB468 cell lysates.

FIG. 2F shows cellular thermal shift assay (CETSA): Band density bar graph comparing stability of the target SHP2 and the non-target HER2 in the JMIT-1 cell lysates.

FIG. 3 shows the effect of BPDA2 on basal ERK1/2 and Akt activation as determined by IB with antibodies that recognize the activated forms (pAkt and pERK) of these proteins, and shows the effect of BPDA2 on the expression of the four RTKs known to be regulated by SHP2.

FIG. 4A shows the effect of BPDA2 on breast cancer cellular phenotypes and that BPDA2 suppresses colony formation in soft agar. B) BPDA2 suppresses mammosphere formation in suspension culture.

FIG. 4B shows the effect of BPDA2 on breast cancer cellular phenotypes and that BPDA2 suppresses mammosphere formation in suspension culture.

FIG. 5 shows the chemical structures of compounds CNBCA and CNBBA of this invention.

FIG. 6A shows a Michaelis-Menten plot showing a shift in DiFMUP concentration (Kₘ) without a change in Vₘₐₓ when CNBCA is added at 1.0 and 2.0 μM concentration. The results are mean±SD of three independent experiments. The insert above the graph shows Pearson Correlation coefficients (r) and p values of X values versus every Y data set.

FIG. 6B shows Lineweaver-Burk plot, confirming an increase in the Km values without affecting the enzyme rate. The insert above the graph shows the slope and the Y intercepts.

FIG. 7A shows Cellular thermal shift assay (CETSA). IB analysis of BT474 lysates treated with 100 μM CNBCA.

FIG. 7B shows Cellular thermal shift assay (CETSA). Bar graph showing the SHP2 and the PTP1B band densities of the IB data from BT474 cell lysates.

FIG. 7C shows Cellular thermal shift assay (CETSA). IB analysis of MDA-MB468 cell lysates treated with CNBCA.

FIG. 7D shows Cellular thermal shift assay (CETSA). Bar graph showing the SHP2 and PTP1B band densities of the IB data from MDA-MB468 cell lysates. The bar graphs were plotted using the Mean±SD of band densities from three independent experiments. FIG. 7E shows Cellular thermal shift assay (CETSA). IB analysis total protein extract (TPE) for expression of FLAG-tagged FL-SHP2 and endogenous EGFR and HER2.

FIG. 7F shows Cellular thermal shift assay (CETSA). IB analysis of FL-SHP2 immunoprecipitated with anti-FLAG antibody.

FIG. 7G shows Cellular thermal shift assay (CETSA). Comparative PTPase assay for activity of FL-SHP2 expressed in MEFs, MDA-MB468, and BT474 cells. The fold relative fluorescence unit (RFU) was calculated by dividing by the background (BG) signal

FIG. 7H shows Cellular thermal shift assay (CETSA). Line graph showing the PTPase assay for determining the effect of CNBCA on FL-SHP2.

FIG. 7I shows Cellular thermal shift assay (CETSA). IB analysis of SHP2 immunoprecipitates for the 12 wells used for the PTPase assay in panel H.

FIG. 8A shows the effect of CNBCA on basal signaling. IB image data showing the effect of CNBCA on basal activation of Akt (pAkt) and ERK1/2 (pERK 1/2) in the BT474 BC cells.

FIG. 8B shows the effect of CNBCA on basal signaling. Band density measurement of pAkt and pERK1/2 in the BT474 cells presented as the ratio of activated over total protein.

FIG. 8C shows the effect of CNBCA on basal signaling. IB image data showing effect of CNBCA on basal activation of Akt (pAkt) and ERK1/2 (pERK1/2) in the MDA-MB468 BC cells.

FIG. 8D shows the effect of CNBCA on basal signaling. Band density measurement of pAkt and pERK 1/2 in the MDA-MB468 cells presented as ratio of activated over total protein. Data are presented as Mean±SD.

FIG. 9A shows the effect of CNBCA on the growth of BT474 breast cancer cells in a 2D culture. Key: V: Vehicle; C: CNBCA.

FIG. 9B shows the effect of CNBCA on the growth of MDA-MB468 breast cancer cells in a 2D culture. Key: V: Vehicle; C: CNBCA.

FIG. 9C shows the effect of CNBCA on colony formation by BT474 breast cancer cells in soft agar. Key: V: Vehicle; C: CNBCA.

FIG. 9D shows the effect of CNBCA on colony formation by MDA-MB468 breast cancer cells in soft agar. Key: V: Vehicle; C: CNBCA.

FIG. 9E shows the effect of CNBCA on mammosphere formation by BT474 breast cancer cells in suspension culture Key: V. Vehicle; C: CNBCA.

FIG. 9F shows the effect of CNBCA on mammosphere formation by MDA-MB468 breast cancer cells in suspension culture. Key: V: Vehicle: C: CNBCA.

### DETAILED DESCRIPTION OF THE INVENTION

This invention provides certain compounds that are phosphotyrosyl phosphatase 2 (SHP2) targeting molecules for use as anti-cancer agents. The compounds of this invention are inhibitors of SHP2. The structures of the compounds of this invention are set forth herein and include BPDA1, BPDA2, BPDA3, BPDA4, BPDA5, BPDA6, BPDA7, BPDA8, BPDA9, and BPDA10 (see Table 1, below).

In certain other embodiments of this invention, compounds that are phosphotyrosyl phosphatase 2 (SHP2) targeting molecules for use as anti-cancer agents are provided, such as CNBCA and CNBBA. The structures of CNBCA and CNBBA are set forth herein.

The present invention provides compounds with increased inhibitory potential and improved selectivity. In certain embodiments of this invention, ten compounds were designed around the CNBDA scaffold, chemically synthesized, and their binding energy and interaction parameters predicted molecular modeling. After confirming structural integrity with NMR and mass spectroscopy, their inhibitory potential and selectivity was tested by a PTPase assay. Among the ten derivatives synthesized, BPDA2 was found to be the most potent and highly selective compound, inhibiting the SHP2 enzyme activity with an IC₅₀ of 92 nM, which is better than the parent compound by approximately 54-fold. Also, BPDA2 is more selective to SHP2 than SHP1 and PTP1B by more than 369-fold and 442-fold, respectively. Evaluation with a cellular thermal shift assay (CETSA) confirmed that BPDA2 binds to wild-type SHP2 in a cellular context and downregulates mitogenic and cell survival signaling and RTK expression. Furthermore, BPDA2 suppresses the anchorage independent growth and cancer stem cell properties of breast cancer cells in a concentration dependent manner. Overall, these findings show that BPDA2 is a more optimized and potent derivative of CNBDA with superior selectivity for SHP2.

The Src homology 2 containing phosphotyrosyl phosphatase 2 (SHP2) is an oncogenic PTP known to promote tumorigenesis in various cancers, including breast cancer (BC). In BC, SHP2 is known to play critical roles in the HER2-positive (HER2+) and the triple-negative breast cancer (TNBC) subtypes, which are both characterized by dysregulation of receptor tyrosine kinase (RTK) signaling [1, 2]. For instance, SHP2 is known to promote epithelial to mesenchymal transition (EMT) [3], cell growth and transformation [4], polarity and migration [5], extracellular matrix degradation and invasion [6], and tumorigenesis and metastasis [1, 7] of BC cells. The cancer-promoting role of SHP2 in BC and other cancers is suggested to be through mediation of RTK signaling. The uniqueness of SHP2 is that its PTPase activity promotes rather than inhibits tyrosine kinase signaling, particularly activation of the Ras-ERK1/2 (extracellular signal regulated kinases 1 and 2) and the PI3K-Akt signaling pathways. The importance of SHP2 in RTK signaling is so critical that effective activation of these signaling pathways cannot occur without it [8-14].

SHP2 is a cytoplasmic Tyr phosphatase with two SH2 domains in the N-terminal and a phosphotyrosyl phosphatase (PTP) domain in the C-terminal regions [15]. While the SH2 domains mediate interaction with phosphotyrosine (pTyr) on RTKs and adaptor proteins, the PTP domain catalyzes dephosphorylation of negative regulatory pTyr sites [16, 17]. For instance, SHP2 dephosphorylates RasGAP (Ras GTPase) docking sites on EGFR (pTyr992) and HER2 (pTyr 1023) to sustain the activated form of Ras (GTP-Ras) [9, 18]. SHP2 has also plays a positive role in signaling by cytoplasmic Tyr kinases such as Src [14]. The activity of SHP2 is regulated by intermolecular and intramolecular interactions. SHP2 assumes an “open conformation” when the SH2 domains interact with pTyr on RTKs and adaptor proteins, leading to its activation to dephosphorylate biological substrates [16, 17]. Therefore, increased Tyr phosphorylation in a tumor cell that engages the SH2 domains such as overexpression of HER2 in HER2+ and multiple other RTKs in TNBC [2, 19, 20] can super-activate SHP2. This is also true in ovarian, lung, and brain cancers, which are characterized by dysregulation of tyrosine kinase signaling Upon disengagement of the SH2 domains. SHP2 folds back and assumes a closed conformation through interaction of the N-SH2 domain with the PTP domain. Mutations that disrupt the closed conformation, particularly mutation of residues that mediate interaction between the N-SH2 and the PTP domain are known to induce constitutive activation of SHP2 [21]. These mutations have been discovered in human diseases particularly in childhood hematopoietic malignancies and physical malformations such as Noonan and LEOPARD syndromes [22].

Recognizing the oncogenic property of SHP2, several groups including us have developed SHP2 inhibitors. Initial efforts focused on development of active-site inhibitors, including PHPS1 [23], SPI-112Me [24], II-B08 [25], 11a-1 [26], 220-32 [27], compound 45 [28], and our compound CNBDA [29]. In the last six years, inhibitors that bind to allosteric sites in SHP2 have also been

developed, including SHP099 and its derivatives [30, 31], and IACS-15414 [32], which showed very promising ant-cancer effects. The major difference between the two classes of inhibitors is that the active site inhibitors bind to the open and active SHP2, while the allosteric inhibitors bind to a closed and inactive SHP2. Since SHP2 has two SH2 (N-SH2 and C-SH2) domains that bind to pTyr and induce an open conformation, the equilibrium between the two states is likely to favor the open conformation under conditions of hyperactive Tyr kinases signaling. This is particularly true in cancers that have dysregulated RTK signaling such as breast (particularly TNBC and HER2+), ovarian, Jung, and brain cancers. SHP2 with an open conformation is least affected by allosteric inhibitors since it loses the major allosteric cleft to which such inhibitors bind. These limitations justify the need to develop active-site inhibitors as well.

Accordingly, we embarked on improving our recently published active site SHP2 inhibitor named CNBDA [29] with the objective to increase inhibitory potential and improve selectivity. In the current study, we report the development of improved active-site SHP2 inhibitors with reduced polarity, increased potency, and superior selectivity. We also report efficacy of the best derivative in inhibiting SHP2-mediated signaling, receptor expression, and breast cancer cell phenotypes.

### Results and Discussion

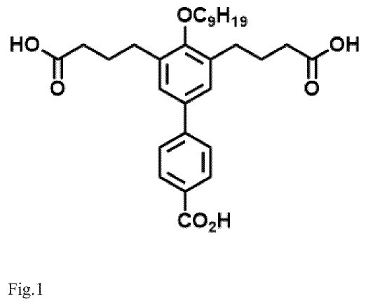

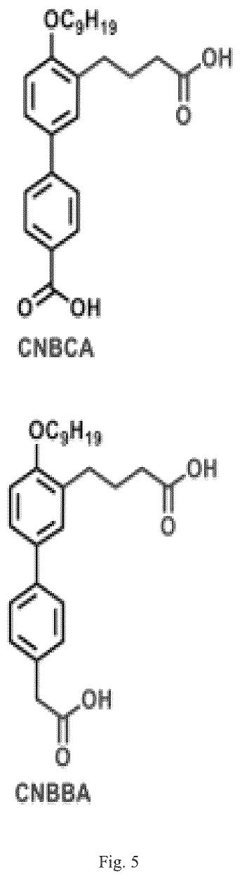

The present invention provides improved derivatives of the parent active-site SHP2 inhibitor (ASSI) named CNBDA [29], which is shown in FIG. 1. Accordingly, we designed ten derivatives that retained the biphenyl core group without the two butyric acid arms. In place of the two butyric acid arms, a single carboxylic group was added in the ortho or meta position of the top phenolic ring to retain binding property. In addition, the acidic group that substitutes the phosphate moiety in phosphotyrosyl (pTyr) substrates was retained as a carboxylic or carboxymethyl group in the para or meta position. We have also retained the aliphatic nolyloxy group that was introduced to enhance cell permeability The chemical name of these derivatives is 4-(nonyloxy)-[1,1′-biphenyl]carboxylic acid (BPDA), and each is identified by a number (1-10) next to the acronym. FIG. 1 shows a structure of the lead (known) compound CNBDA. FIG. 5 shows the structures of CNBCA and CNBBA.

As an initial approach, we conducted molecular docking studies to predict the interaction properties and binding energies of the new compounds, using the molecular modeling program Glide (Schrodinger) followed by an induced-fit docking and binding energy calculations with Prime MM-GB/SA [33]. Each of the designed molecules were docked into the SHP2 active site PDB:4DGP as reported previously [29, 34]. We also docked these structures into the SHP1 active site (PDB: 1GWZ), the close homolog of SHP2, for the purpose of comparison Levels of predicted free energies (ΔG≥−40 Kcal/mol) and differences in binding energies between SHP2 and SHP1 (ΔG≥−20 Kcal/mol) are provided in Table 1. As shown, all ten compounds bind to the SHP2 active with significantly higher energies (ΔGs) than to the SHP1 active site. Based on these observations, the 10 compounds were processed for the next step—chemical synthesis.

TABLE 1

| Structure | 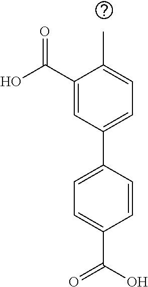 [SMILES: Cc1ccc(-c2ccc(C(=O)O)cc2)cc1C(=O)O] | 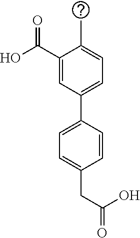 [SMILES: Cc1ccc(-c2ccc(CC(=O)O)cc2)cc1C(=O)O] | 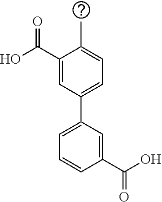 [SMILES: Cc1ccc(-c2cccc(C(=O)O)c2)cc1C(=O)O] | 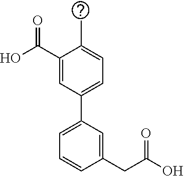 [SMILES: Cc1ccc(-c2cccc(CC(=O)O)c2)cc1C(=O)O] | 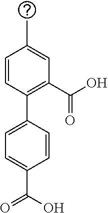 [SMILES: Cc1ccc(-c2ccc(C(=O)O)cc2)c(C(=O)O)c1] |
|---|---|---|---|---|---|
| Name | BPDA1 | BPDA2 | BPDA3 | BPDA4 | BPDA5 |
| ΔG SHP2 | −39.7 | −52.31 | −41.84 | −47.02 | −40.59 |
| ΔG SHP1 | −14.58 | −16.64 | −10.99 | −13.3 | −10.7 |
| Difference | −24.87 | −35.67 | −30.85 | −33.72 | −29.89 |
| Structure | 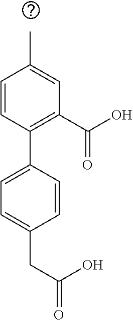 | 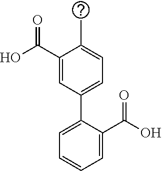 | 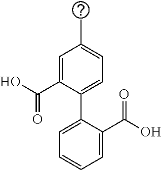 | 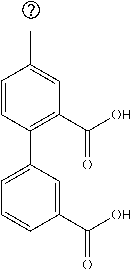 | 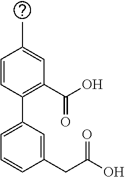 |
| Name | BPDA6 | BPDA7 | BPDA8 | BPDA9 | BPDA10 |
| ΔG SHP2 | −46.08 | −49.73 | −38.8 | −44.16 | −41.29 |
| ΔG SHP1 | −10.99 | −22.17 | −22.93 | −26.18 | −12.4 |
| Difference | −35.09 | −27.56 | −15.87 | −17.98 | −28.89 |

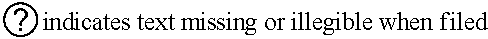

Table 1: Shows results of docking the 10 CNBDA derivatives (compounds of this invention) into the SHP2 and SHP1 active sites. Also shown are the differences in the predicted ΔG of binding between SHP2 and SHP1 for each compound.

Because of the differences in the positions of the R groups (see Table 2) and the corresponding starting materials, we used different routes to synthesize the ten compounds. The details of the synthesis routes for the 4-(nonyloxy)-[1,1′-biphenyl]carboxylic acid (BPDA1-7) derivatives are outlined in Scheme 1. BPDA1 and BPDA2 were synthesized using scheme 1a, BPDA3 and BPDA4 were synthesized using scheme 1b, BPDA5 and BPDA6 were synthesized using scheme 1c, BPDA7 was synthesized using scheme 1d, BPDA8 was synthesized using scheme 1e, and BPDA9 and BPDA10 were synthesized scheme 1f. The details of the synthesis route and conditions are provided in the experimental section.

The synthesis of BPDA1 was started by reacting S-Bromo-2-hydroxybenzoic acid methyl ester (1a) with 4-(methoxycarbonyl) phenylboronic acid (2c), under optimized Suzuki-cross coupling conditions to produce biphenyl intermediate 3c with 50% yield. This intermediate was then subjected to O-alkylation with 1-bromononane, using K₂CO₃ in acetonitrile to produce 4c with 75% yield. In the end, the di-ester hydrolysis was achieved using LiOH·H₂O in THF:H₂O (1:1) at room temperature to produce BPDA1 (5c) with 89% yield (Scheme 1a). The formation of BPDA1 was confirmed by NMR and HRMS analysis.

The synthesis BPDA2 was started by reacting compound 1a with 4-(4,4,5,5-Tetramethyl-1,3,2-dioxaborolan-2-yl)benzeneacetonitrile (2d), which resulted in an intermediate 3d with 52% yield. The 3d intermediate was then alkylated with 1-bromononane to give 4d. Hydrolysis of methyl ester and cyanomethyl group was accomplished using NaOH in ethanol:water (1:1) refluxed for 16 h to furnish BPDA2 (5d) with 80% yield (Scheme 1a).

BPDA3-10 were synthesized using the indicated starting materials and the differently substituted phenyl boronic acid or pinacol-ester, following similar synthetic sequences to the Suzuki coupling, O-alkylation, and hydrolysis. The Suzuki-cross coupling reaction was optimized for the synthesis of intermediate 3f, 3h, 3i, 3j, 3k and 3l by using Pd(PPh₃)₄, Cs₂CO₃ in THF (Scheme 1b, 1c, 1d).

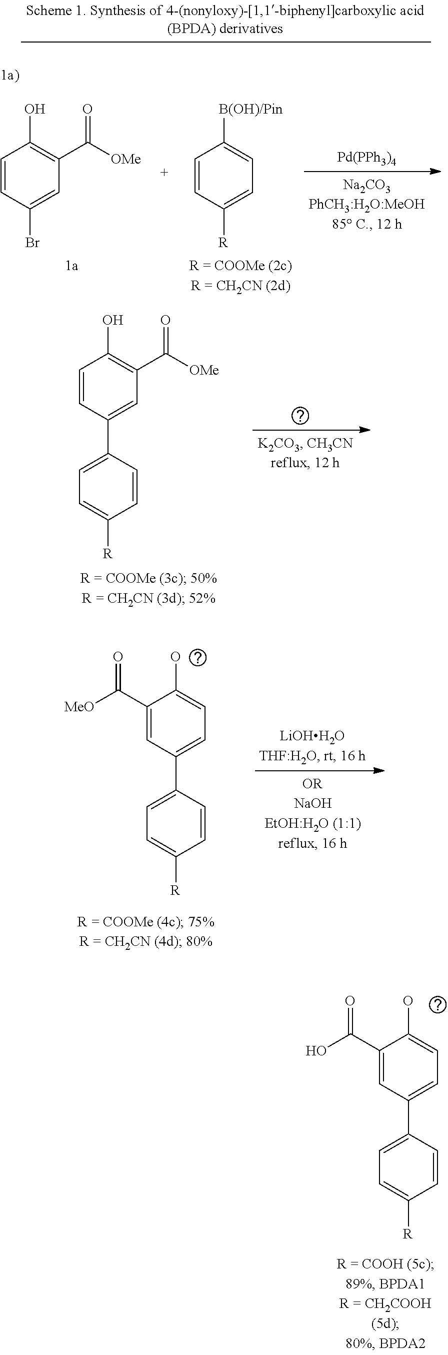

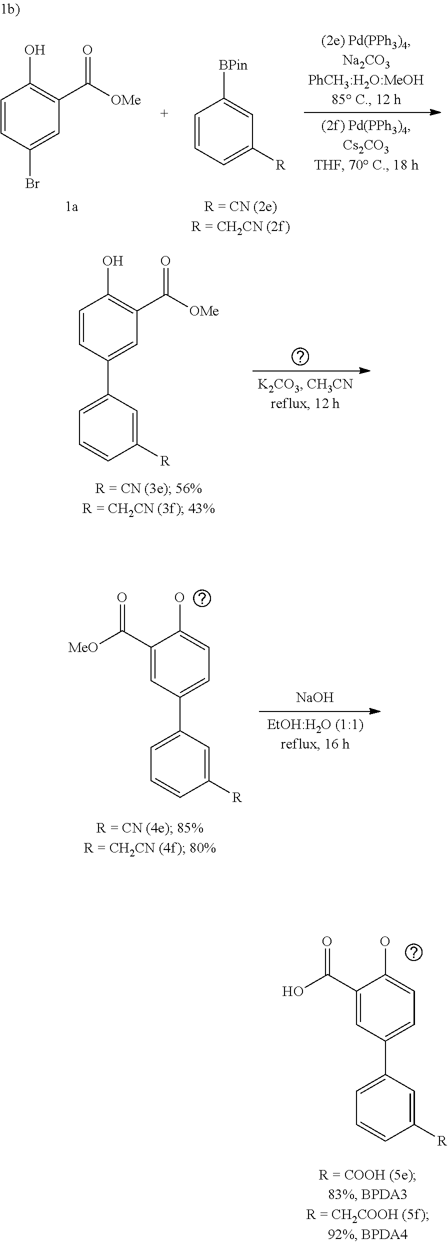

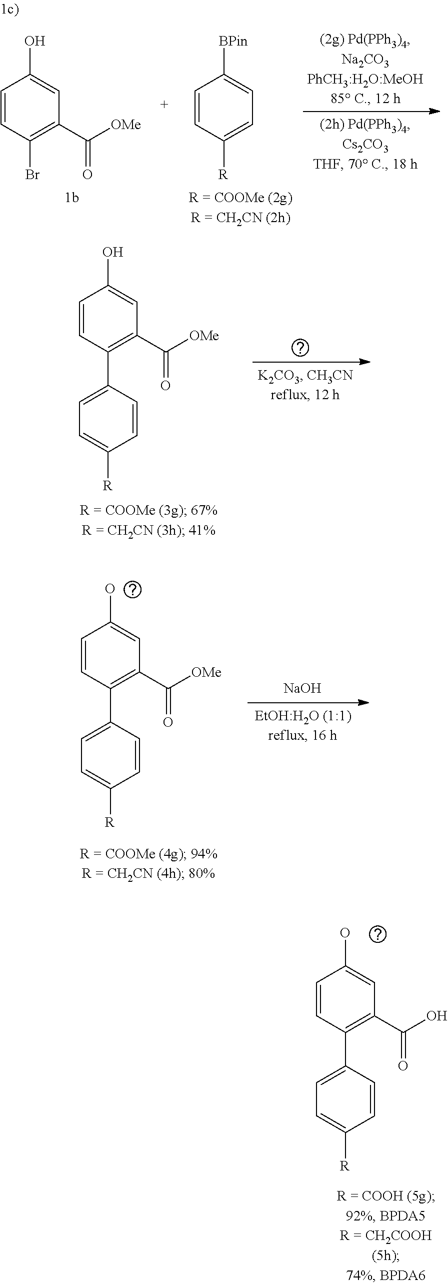

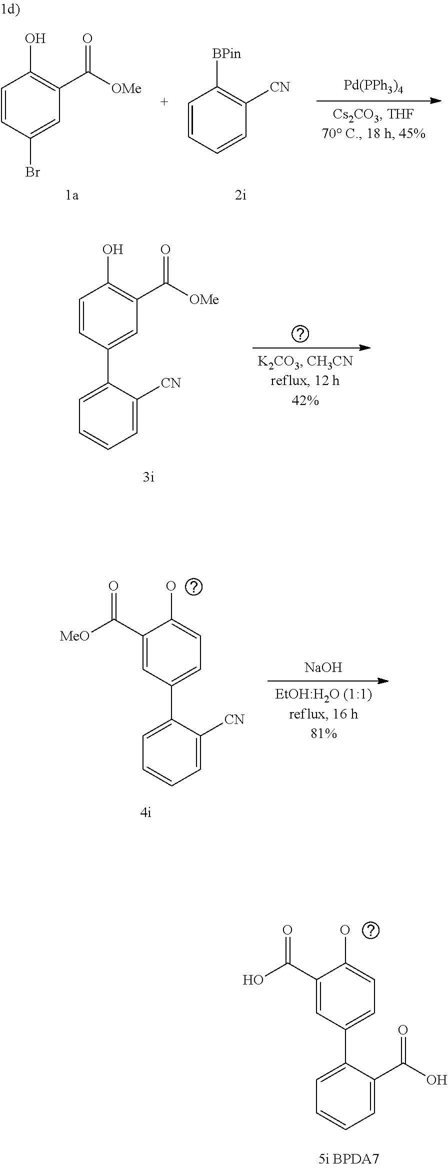

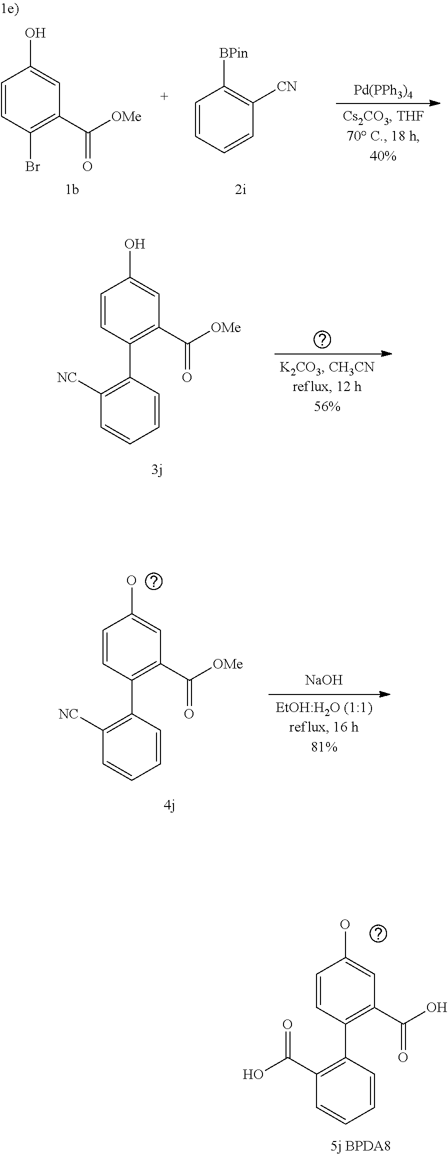

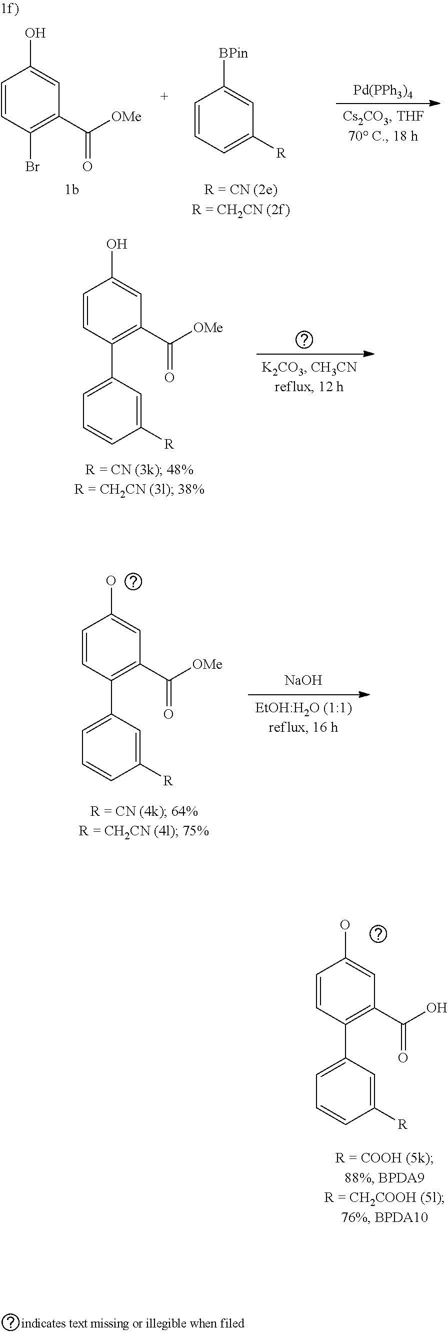

SAR evaluation on enzyme activity and selectivity. After confirming the mass and structural integrity of each of the ten compounds, we performed structure-activity relationship (SAR) studies using PTPase assays to evaluate the effect of each compound on the SHP2 enzyme activity. Briefly, the inhibitors were added first in a serial dilution (12 nM-100 μM), followed by the purified PTP domain of SHP2 at 1.0 nM, and finally the artificial substrate DiFMUP to a final concentration of 20 μM in a reaction volume of 100 μl. After incubation at 37° C. for 20 minutes, fluorescence intensity of the reactions was measured by the Synergy 4 plate reader at the excitation and emission wave lengths of 360 and 460 nm, respectively. The IC₅₀ of each compound was determined by employing the GraphPad software. The results showed inhibition of the SHP2 enzyme activity by each molecule with variable IC₅₀, ranging from 92 nM to 29.7 μM (Table 2). Of the seven derivatives, BPDA2 showed the best IC₅₀, which is 92 nM, followed by BPDA6, which is 216 nM. When compared to the parent compound CNBDA that showed an IC₅₀ of 5 μM [29], BPDA2 and BPDA6 showed more than 54 and 23 times better IC₅₀, respectively. These findings show that we have produced potent derivatives of the parent compound.

Table 2 shows R group substitutes to the parent compound CNBDA and the corresponding IC₅₀ values for inhibiting the SHP2 PTPase activity. The parent compound scaffold is shown below and the compounds BPDA1 thru BPDA10 of this invention are shown in Table 2:

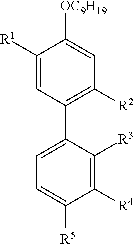

TABLE 2

| Compound | R¹ | R² | R³ | R⁴ | R⁵ | IC₅₀ (μM) |
|---|---|---|---|---|---|---|
| BPDA1 | COOH | H | H | H | COOH | 9.50 |
| BPDA2 | COOH | H | H | H | CH₂COOH | 0.092 |
| BPDA3 | COOH | H | H | COOH | H | 28.29 |
| BPDA4 | COOH | H | H | CH₂COOH | H | 11.70 |
| BPDA5 | H | COOH | H | H | COOH | 29.70 |
| BPDA6 | H | COOH | H | H | CH₂COOH | 0.216 |
| BPDA7 | COOH | H | COOH | H | H | 7.00 |
| BPDA 8 | H | COOH | COOH | H | H | 18.70 |
| BPDA9 | H | COOH | H | COOH | H | 2.30 |
| BPDA 10 | H | COOH | H | CH2COOH | H | 1.80 |

We compared the selectivity of the best derivatives, BPDA2 and BPDA6, using the PTPase assay. For these comparisons, we used the close structural homolog SHP1 and the ubiquitously expressed PTP1B, which are both negative regulators of receptor tyrosine kinase (RTK) signaling as opposed to SHP2, which is a positive regulator. The effect of the compounds was tested using purified PTP domains of both SHP1 and PTP1B under same conditions described for SHP2, but using DiFMUP concentrations that reflect the Km values for each PTP (see experimental section). As shown in Table 3, BPDA2 is less effective against SHP1 and PTP1B as evidenced by the corresponding IC₅₀ values, which were 33.39 μM and 40.71 μM, respectively. Calculation of selectivity ratios suggested that BPDA2 is more selective for SHP2 than for SHP1 and PTP1B by more than 362 and 442 fold, respectively. BPDA6 also was less effective against SHP1 and PTP1B. More specifically, BPDA6 inhibited SHP1 and PTP1B with an IC₅₀ of 10 μM and 11.8 μM, respectively. Selectivity ratio calculations for BPDA6 showed that it is also more selective for SHP2 than for SHP1 and PTP1B by more than 46 and 54 times, respectively. These findings show that BPDA2 and BPDA6 are highly selective to SHP2 than to SHP1 and PTP1B with BPDA2 being the most selective and potent inhibitor.

Our parent compound CNBDA was selective to SHP2 than to SHP1 by approximately 25 fold [29]. As mentioned above, the new derivatives of CNBDA, particularly BPDA2 is more than 369-fold and 442-fold selective for SHP2 than for SHP1 and PTP1B, respectively (Table 3). These findings suggest that we have created more selective active-site SHP2 inhibitors than the parent compound. In addition, our results show that BPDA2 is more comparable to the previously reported active site inhibitors, particularly 11a-1 which showed an IC₅₀ of 200 nM [26] and compound 45 which showed an IC₅₀ of 31 nM [28], in inhibiting SHP2 enzyme activity.

Table 3 compares the selectivity of BPDA2 and BPDA6 to the three PTPs.

TABLE 3

| Compound | PTP | IC₅₀ (μM) | Selectivity ratio |
|---|---|---|---|
| BPDA2 | SHP2 | 0.092 | 1.0 |
| | SHP1 | 33.39 | 362.9 |
| | PTP1B | 40.71 | 442.4 |
| BPDA6 | SHP2 | 0.216 | 1.0 |
| | SHP1 | 10.00 | 46.29 |
| | PTP1B | 11.80 | 54.6 |

BPDA2 confers stability of the SHP2 protein in a cellular context. The SAR and the selectivity data presented in Table 2 and Table 3, respectively, show that BPDA2 is the best active-site SHP2 inhibitor among the ten derivatives. Here, we wanted to confirm whether BPDA2 binds to full-length SHP2 in a cellular context. We used the cellular thermal shift assay (CETSA) combined with immunoblotting (IB) to determine target engagement. The CETSA assay is based on the principle that drug binding to a target protein confers stability from heat-induced unfolding and precipitation when compared to the non-drug bound counterpart [35]. Total cell lysates (TCL) prepared from two breast cancer cell lines, the triple-negative MDA-MB468 and the HER2-positive JIMT-1 cells, were cleared by centrifugation, mixed with BPDA2 at 100 μM (assuming similar SHP2 level in cells), incubated at room temperature for 10 minutes to allow binding, divided into 10 aliquots, and then treated with heat, ranging from 37° C. to 64° C. that differ by 3° C. intervals, for 10 minutes. Non-BPDA2 treated samples were used as controls. After heat treatment, samples were centrifuged at 20,000 g for 20 minutes at 4° C. to remove precipitated proteins, and supernatants were analyzed by IB for SHP2. We also analyzed for EGFR in the MDA-MB468 and for HER2 in the JIMT-1 samples as non-target proteins to show selectivity in heat stabilization.

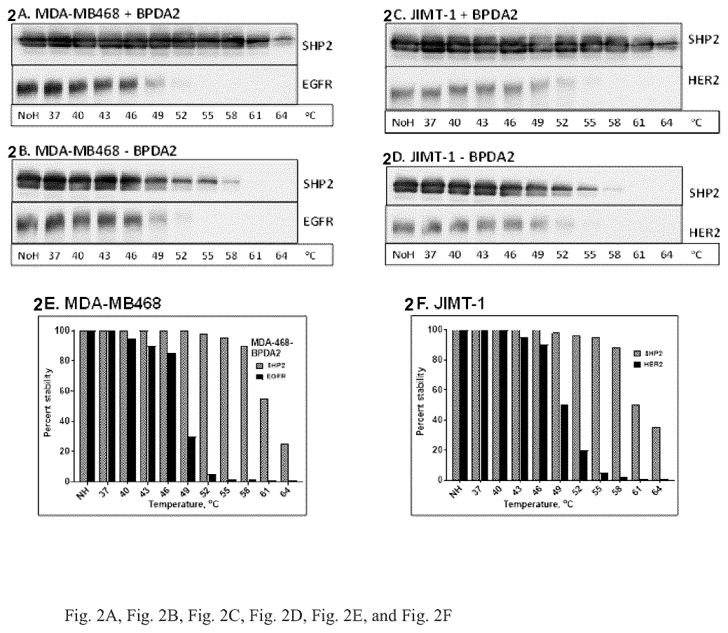

The CETSA results showed that BPDA2 treatment selectively stabilizes SHP2 in solution when compared to the non-target proteins EGFR and HER2 (FIGS. 2A and C). In the absence of BPDA2 treatment, stability of the SHP2 protein in solution was similar to that of the non-target proteins EGFR and HER2 (FIGS. 2B and D). On the other hand, the non-target EGFR and HER2 proteins were reduced below 50% at 46° C. and at 49° C., respectively, and became undetectable at 55° C. (FIGS. 2A and C). In the absence of BPDA2 treatment, the stability of SHP2 in both cell types was similar to that of the non-target EGFR and HER2 proteins (FIGS. 2B and D). Band density measurements confirmed that BPDA2 stabilized SHP2 in solution to nearly 100% at 49° C. and to 85% at 58° C. (FIGS. 2E and F). The percent stability of the non-target proteins EGFR and HER2 at 58° C. was virtually undetectable. These findings suggest that BDPA2 selectively binds to SHP2, which in turn leads to resistance to heat-induced unfolding and precipitation. As such, it is reasonable to conclude that BPDA2 engages SHP2 in cellular contexts.

FIG. 2A shows cellular thermal shift assay (CETSA of MDA-MB468 lysates treated with BPDA2. FIG. 2B shows MDA-MB468 cell lysates not treated with BPDA2 FIG. 2C shows JIMT-1 cell lysates treated with BPDA2. FIG. 2D shows JIMT-1 cell lysates not treated with BPDA2. FIG. 2E shows Band density bar graph comparing stability of the target SHP2 and the non-target EGFR in the MDA-MB468 cell lysates. FIG. 2F shows Band density bar graph comparing stability of the target SHP2 and the non-target HER2 in the JMIT-1 cell lysates.

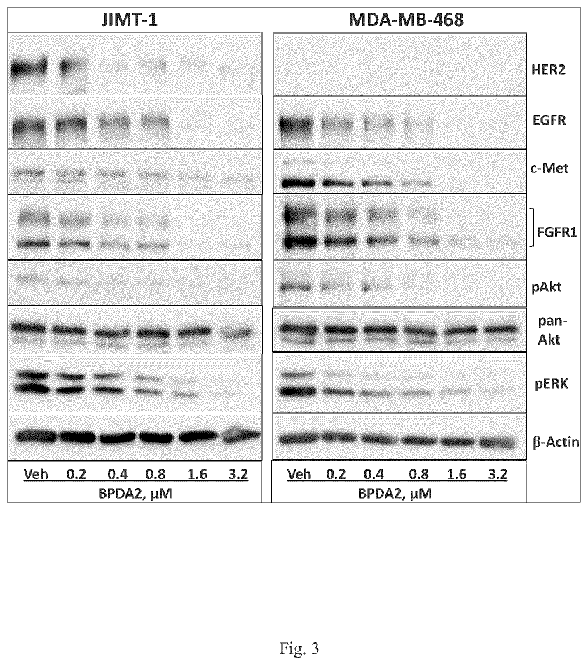

BPDA2 inhibits SHP2-mediated signaling Since SHP2 is a master regulator of signaling, we treated cells with variable concentrations of BPDA2, ranging from 0.2-3.2 μM, in a regular growth medium, and determined effect on Akt and ERK1/2 activation by IB with antibodies that detect the activated forms of these proteins. As shown in FIG. 3, BPDA2 inhibited basal activation of Akt and ERK 1/2 in a concentration dependent manner. Consistent with our previous reports, inhibition of SHP2 with BPDA2 downregulated the expression of the four RTKs in a concentration dependent manner. These findings suggest that targeting SHP2 with BPDA2 inhibits basal signaling and RTK expression in breast cancer cells, which is consistent with prior reports by us that used various technics to inhibit SHP2 [1, 29, 36]. Also, our results are consistent with previous reports by others that showed suppression of signaling by targeting SHP2 with active-site inhibitors [26, 28].

FIG. 3 shows the effect of BPDA2 on basal ERK1/2 and Akt activation as determined by IB with antibodies that recognize the activated forms (pAkt and pERK) of these proteins. Also shown is effect of BPDA2 on the expression of the four RTKs known to be regulated by SHP2.

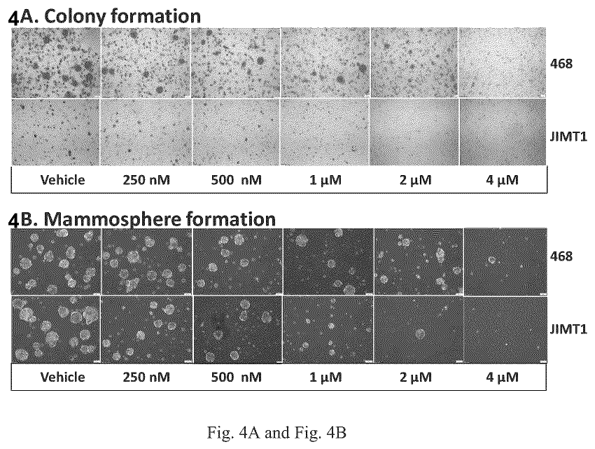

BPDA2 suppresses anchorage-independent growth and cancer stem cell properties. Previous reports by us and others have shown that inhibiting SHP2 by various means, such as shRNA-mediated silencing, dominant-negative expression or pharmacological targeting in breast cancer cells blocks colony formation in soft agar and mammosphere formation in suspension culture [1, 29]. We used these assays to determine effect of SHP2 targeting with BPDA2 on cell transformation and cancer stem cell (CSC) properties, respectively. To determine effect on anchorage-independent growth, approximately 10⁶ JIMT-1 and MDA-MB468 cells were seeded in soft agar in 6 cm plates and then treated with vehicle or six different concentrations of BPDA2, ranging from 250 nM to 4.0 μM. The results showed suppression of colony formation by BPDA2 in a concentration dependent manner (FIG. 4A). These findings suggest that BPDA2 suppresses colony formation by the HER2-positive and triple-negative breast cancer cells.

To determine the effect of BPDA2 on CSC properties, approximately 10⁶ cells were seeded in non-adherent 6 cm plates in suspension cultures, in which only cells with stem-like properties can grow and form mammospheres, which are also known as tumoripheres in other cancer types. While the vehicle-treated cells formed larger and relatively several mammosphere, the BPDA2-treated cells formed fewer and smaller ones as the concentration increased (FIG. 4B). Hence, inhibition of SHP2 with BPDA2 eliminates the CSCs in the HER2-positive JIMT-1 and the triple-negative MDA-MB468 cells.

FIG. 4A shows the effect of BPDA2 on breast cancer cellular phenotypes and that BPDA2 suppresses colony formation in soft agar. FIG. 4B shows that BPDA2 suppresses mammosphere formation in suspension culture.

Those persons of ordinary skill in the art will understand that the present invention, guided by molecular modeling, provides for the synthesis of novel compounds that are derivatives of the parent compound CNBDA [29] with the objective to provide novel compounds having increased inhibitory potential and improved selectivity without compromising efficacy. Among the ten derivatives, BPDA2 was found to be the most potent and highly selective compound. More specifically, BPDA2 inhibited the SHP2 enzyme activity with an IC₅₀ of 92 nM, which is better than the parent compound by approximately 54-fold. Also, BPDA2 is more selective to SHP2 than to SHP1 and PTP1B by more than 369-fold and 442-fold, respectively. CETSA analysis confirmed that BPDA2 binds to wild-type SHP2 in a cellular context and downregulates mitogenic and cell survival signaling and RTK expression. Furthermore, BPDA2 suppresses the anchorage independent growth and cancer stem cell properties of breast cancer cells in a concentration dependent manner. Overall, these findings suggest that BPDA2 is a highly optimized and potent derivative of CNBDA with superior selectivity for SHP2. However, our findings cannot fully exclude the possibility of BDPA2 inhibiting other PTPs.

### Experimental Section I

The present invention is more particularly described in the following non-limiting examples, which are intended to be illustrative only.

Molecular docking. To predict the binding properties of the 10 CNBDA derivatives, we used the molecular docking program Glide (by Schrodinger) that allows receptor preprocessing, ligand preparation, molecular docking, interaction mapping, and binding energy calculations (MM-GB/SA). Docking the newly designed compounds into the SHP2 and SHP1 active sites was performed as described by us recently [29]. Briefly, the structures of the 10 compounds were docked into the SHP2 (PDB: 4DGP) and the SHP1 (PDB: 1GWZ) active sites after loading, processing, and optimizing using the Epik program in the Glide software package. Since the SHP2 structure PDB:4DGP was solved with both of the SH2 domains, it was necessary to remove the N-terminal SH2 (N-SH2) domain from the structure to allow docking. But, such processing was unnecessary for SHP1 since PDB:1GWZ contained only the PTP domain. Since the compounds are designed to bind to the active site of SHP2, 20×20×20 Å grid box was placed around the point defined by the sulfur atom of the catalytic cysteine (C459 for SHP2 and C455 for SHP1). All of the 10 compounds were built in Chemdraw, saved as mol files, and docked into the active sites of SHP2 and SHP1 defined by the grid box. Prime MM-GBSA calculations were used to predict changes in free energy of binding denoted as ΔG. The 2-dimensional interaction diagram was generated from the PDB file of the docked poses, using the MAESTRO 2-D sketcher of Glide.

Chemistry. For organic synthesis, reagents and solvents were purchased from (Sigma Aldrich, TCI, Oakwood, Enamine, and ChemImpex) and it was used without additional purifications. ¹H and ¹³C NMR spectra were recorded on Geol 400 spectrometers with TMS or residual solvent as standard. Column chromatography was performed on silica gel (100-200 mesh) using a proper eluent. Thin-layer chromatography (TLC) analysis was performed on precoated silica gel 60 F254 plates. Visualization on TLC was achieved by the use of UV light (254 nm). NMR spectra were recorded in chloroform-d, Methanol-d₄ and DMSO-d₆ at 400 MHz for ¹H NMR spectra and 100 MHz ¹³C NMR spectra. Chemical shifts were quoted in parts per million (ppm) referenced to the appropriate solvent peak or 0.0 ppm for tetramethylsilane. The following abbreviations were used to describe peak splitting patterns when appropriate: br=broad, s=singlet, d=doublet, t=triplet, q=quartet, p=pentet (quintet), dd=doublet of doublet, td=triplet of doublet, m=multiplet. Coupling constants (J) are reported in hertz (Hz). ¹³C NMR chemical shifts were reported in ppm referenced to the center of a triplet at 77.0 ppm of chloroform-d, 49.0 ppm for methaon-d₄ and 40.0 ppm center for DMSO-d₆. HRMS spectra were recorded using Quadrupole and Orbitrap LC-MS/MS techniques. The purity of the compounds was determined by HPLC, and all of them are more than 95% pure.

Dimethyl 4-hydroxy-[1,1′-biphenyl]-3,4′-dicarboxylate (3c). A modified Suzuki-cross coupling protocol was used to synthesize the compounds [37]. Under nitrogen atmosphere, a mixture of 5-Bromo-2-hydroxybenzoic acid methyl ester (1a, 500 mg, 2.16 mmol), 4-(methoxycarbonyl) phenylboronic acid (2c, 468, 2.6 mmol), Pd(PPh₃)₄ (125 mg, 5 mol %), Na₂CO₃ (458 mg, 4.32 mmol) in toluene (5 mL), water (2.5 mL), and methanol (2.5 mL) was refluxed for 12 h at 85° C. After the completion of the reaction (by TLC), the mixture was cooled to room temperature, the solvent was evaporated, and the crude residue was extracted with ethyl acetate (50 mL×3). The extract was washed with water and brine, dried over Na₂SO₄, and evaporated. The residue was purified by column chromatography over silica gel (n-hexane/ethyl acetate=1:1) to give 3c as a white solid (310 mg, 50%). The spectroscopic data is consistent with the reported literature[38] ¹H NMR (400 MHz, CDCl₃) δ 10.84 (s, 1H), 8.09 (m, J=12.5, 9.8 Hz, 4H), 7.76-7.64 (m, 2H), 7.60 (d, J=8.2 Hz, 2H), 7.07 (d, J=8.7 Hz, 1H), 3.98 (s, 3H), 3.93 (s, 3H); ¹³C NMR (100 MHz, CDCl₃) δ 170.3, 166.8, 161.6, 144.1, 134.3, 131.0, 130.1, 129.9, 128.6, 128.4, 127.2, 126.3, 118.2, 112.6, 52.4, 52.0.

Dimethyl 4-(nonyloxy)-[1,1′-biphenyl]-3,4′-dicarboxylate (4c). Under nitrogen atmosphere, a mixture of 3c (75 mg, 0.262 mmol), K₂CO₃ (73 mg, 0.524 mmol) in anhydrous acetonitrile 5 mL was stirred at rt for 10 min, to that 1-Bromononane (68 mg, 0.328 mmol) was added and resulting mixture was refluxed for 12 h. After completion by TLC, mixture was cooled down to room temperature and then diluted with water and extracted with ethyl acetate (50 mL×2). The extract was washed with water and brine, dried over Na₂SO₄, and evaporated. The crude oil was purified by column chromatography over silica gel (n-hexane/ethyl acetate, 8:2) to give 4c as a white solid (82 mg, 75%). ¹H NMR (400 MHz, CDCl₃) δ 8.08 (m, J=8.4 Hz, 3H), 7.70 (d, J=8.7 Hz, 1H), 7.66-7.59 (m, 2H), 7.04 (d, J=8.7 Hz, 1H), 4.08 (t, J=6.4 Hz, 2H), 3.97-3.89 (2s, 6H), 1.85 (p, J=6.7 Hz, 2H), 1.58-1.43 (p, 2H), 1.33 (m, J=29.1 Hz, 10H), 0.88 (t, J=6.7, 5.7 Hz, 3H). ¹³C NMR (100 MHz, CDCl₃) δ 166.9, 166.7, 158.7, 144.1, 131.8, 131.6, 130.3, 130.1, 128.6, 126.4, 120.9, 113.6, 69.1, 52.0, 52.0, 31.8, 29.5, 29.3, 29.2, 29.1, 25.9. 22.6, 14.0.

4-(nonyloxy)-[1,1′-biphenyl]-3,4′-dicarboxylic acid (5c, BPDA1). Compound 4c (60 mg, 0.145 mmol) in THF (2 mL) was added to a solution of LIOH·H₂O (19 mg, 0.452 mmol) in water (2 mL). The resulting mixture was stirred at room temperature for 16 h, and when TLC (50% ethyl acetate/hexane) showed that the reaction was completed, the mixture was concentrated under reduced pressure to remove tetrahydrofuran. The resulting aqueous solution was cooled in an ice bath and made acidic (pH 1) with 1 N HCl to give a fine white precipitate. The aqueous suspension was extracted with an ethyl acetate (25 mL×3). The organic phase was washed with saturated brine (50 mL), dried over sodium sulfate, and filtered, and the filtrate was concentrated under reduced pressure to give a white solid. It was purified by column chromatography over silica gel (n-hexane/ethyl acetate, 4:6) to give 5c a pure white solid (50 mg, 89%). ¹H NMR (400 MHz, DMSO-D₆) δ 12.85 (s, 1H), 8.00 (d, J=8.2 Hz, 2H), 7.95 (d, J=1.1 Hz, 1H), 7.86 (d, J=8.7 Hz, 1H), 7.77 (d, J=8.1 Hz, 2H), 7.22 (d, J=8.8 Hz, 1H), 4.07 (t, J=6.2 Hz, 2H), 1.79-1.65 (p, 2H), 1.43 (p, J=13.9, 6.5 Hz, 2H), 1.27 (m, J=22.7 Hz, 10H), 0.85 (t, J=6.5 Hz, 3H). ¹³C NMR (100 MHz, DMSO-D₆) δ 167.7, 167.7, 167.6, 158.0, 158.0, 143.6, 143.6, 131.6, 131.6, 131.0, 131.0, 130.5, 130.5, 129.6, 129.3, 126.7, 126.7, 122.8, 114.5, 68.9, 68.9, 39.8, 39.8, 39.6, 39.6, 39.3, 39.3, 31.8, 31.8, 29.5, 29.5, 29.2, 29.1, 29.1, 29.1, 25.9, 25.9, 22.6, 22.6, 14.4. HRMS (ESI) [M−H]⁻: m/z caldt for C₂₃H₂₈O₅: 383.1864, found: 383.1864.

Methyl 4′-(cyanomethyl)-4-hydroxy-[1,1′-biphenyl]-3-carboxylate (3d). According to the procedure described for the preparation of 3c, the same procedure was followed for 3d; Under nitrogen atmosphere, 5-Bromo-2-hydroxybenzoic acid methyl ester (1a, 500 mg, 2.16 mmol), 4-(4,4,5,5-Tetramethyl-1,3,2-dioxaborolan-2-yl)benzeneacetonitrile (2d) 630 mg, 2.59 mmol), Pd (PPh₃)₄ (125 mg, 5 mol %), Na₂CO₃ (458 mg, 4.32 mmol), in toluene (5 mL), water (2.5 mL) and methanol (2.5 mL) was refluxed for 12 h at 85° C. to yield as white solid 300 mg (52%). ¹H NMR (400 MHz, CDCl₃) δ 10.80 (s, 1H), 8.06 (s, 1H), 7.69 (d, J=8.7 Hz, 1H), 7.56 (d, J=8.0 Hz, 2H), 7.39 (d, J=7.9 Hz, 2H), 7.07 (d, J=8.6 Hz, 1H), 3.99 (s, 3H), 3.80 (s, 2H). ¹³C NMR (100 MHz, CDCl₃) δ 170.4, 161.2, 139.8, 134.2, 131.4, 128.6, 128.4, 128.1, 127.3, 118.2, 117.78, 112.6, 52.4, 23.3.

Methyl 4′-(cyanomethyl)-4-(nonyloxy)-[1,1′-biphenyl]-3-carboxylate (4d). According to the procedure described for the preparation of 4c, the same procedure was followed for 4d; Under nitrogen atmosphere, a mixture of 3d (185 mg, 0.690 mmol), K₂CO₃ (190 mg, 0.940 mmol) in anhydrous acetonitrile 10 mL was stirred at room temperature for 10 min, to that 1-Bromononane (171 mg, 0.831 mmol) was added and resulting mixture was refluxed for 12 h; obtained 4d as a light yellow solid (220 mg, 80%). ¹H NMR (400 MHz, CDCl₃) δ 8.01 (d, J=2.4 Hz, 1H), 7.65 (dd, J=8.6, 2.4 Hz, 1H), 7.58 (d, J=8.2 Hz, 2H), 7.39 (d, J=8.1 Hz, 2H), 7.04 (d, J=8.7 Hz, 1H), 4.08 (t, J=6.5 Hz, 2H), 3.92 (s, 3H), 3.79 (s, 2H), 1.92-1.76 (p, 2H), 1.49 (m, J=15.0, 7.3 Hz, 2H), 1.36-1.24 (m, 10H), 0.89 (t, J=6.7 Hz, 3H). ¹³C NMR (100 MHz, CDCl₃) δ 166.9, 158.3, 139.8, 132.0, 131.6, 130.1, 129.8, 128.6, 128.4, 127.3, 120.84, 117.8, 113.7, 69.2, 52.0, 31.9, 29.5, 29.3, 29.2, 29.1, 25.9, 23.3, 22.7, 14.0.

4′-(carboxymethyl)-4-(nonyloxy)-[1,1′-biphenyl]-3-carboxylic acid (5d, BPDA2). To a solution of NaOH (65 mg, 1.62 mmol) in water (3 mL) was added a solution of compound 4d (160 mg, 0.407 mmol) in EtOH (3 mL). The resulting mixture was refluxed for 16 h, and when TLC (60% ethyl acetate/hexane) showed that the reaction was completed, the mixture was concentrated under reduced pressure to remove EtOH. The resulting aqueous solution was cooled in an ice bath and made acidic (pH 1) with 1 N HCl to give a precipitate. The aqueous suspension was extracted with an ethyl acetate (40 mL×3). The organic phase was washed with saturated brine (50 mL), dried over sodium sulfate, and filtered, and the filtrate was concentrated under reduced pressure to give a white solid. It was purified by column chromatography over silica gel (n-hexane/ethyl acetate, 3:7) to give 5d as a white solid (130 mg, 80%). ¹H NMR (400 MHz, CDCl₃) δ 8.41 (s, 1H), 7.75 (d, J=8.6 Hz, 1H), 7.55 (d, J=7.5 Hz, 2H), 7.36 (d, J=7.6 Hz, 2H), 7.11 (d, J=8.6 Hz, 1H), 4.28 (t, J=6.4 Hz, 2H), 3.70 (s, 2H), 1.98-1.86 (p, 2H), 1.56-1.44 (m, 2H), 1.29 (m, J=4.4 Hz, 10H), 0.89 (t, J=6.3 Hz, 3H). ¹³C NMR (101 MHz, CDCl₃) δ 165.7, 157.0, 138.1, 134.6, 133.5, 133.2, 132.2, 132.6, 131.9, 131.0, 129.9, 127.0, 117.9, 113.1, 70.4, 40.5, 31.8, 29.3, 29.2, 29.1, 28.9, 25.8, 22.6, 14.0. HRMS (ESI) [M+Na]⁺: m/z caldt for C₂₄H₃₀O₅: 421.20; found: 421.20.

Methyl 3′-cyano-4-hydroxy-[1,1′-biphenyl]-3-carboxylate (3e). The same protocol described for the 3c was followed for 3e. 5-Bromo-2-hydroxybenzoic acid methyl ester (1a, 500 mg, 2.16 mmol), (3-Cyanophenyl)boronic acid pinacol ester (2e) 594 mg, 2.59 mmol), Pd(PPh₃)₄ (125 mg, 5 mol %), Na₂CO₃ (458 mg, 4.32 mmol) in toluene (5 mL), water (2.5 mL), and methanol (2.5 mL) was refluxed for 12 h at 85° C. under nitrogen atmosphere to obtain 3e as half white solid of 307 mg (56%). ¹H NMR (400 MHz, methanol-d₄) δ 8.01 (d, J=8.0 Hz, 2H), 7.33 (d, J=8.1 Hz, 2H), 7.24 (d, J=8.4 Hz, 1H), 7.21 (d, J=1.9 Hz, 1H), 7.01 (dd, J=8.3, 2.1 Hz, 1H), 3.61 (s, 3H). ¹³C NMR (100 MHz, methanol-d₄) δ 170.5, 169.8, 158.5, 147.6, 133.7, 133.1, 133.0, 130.5, 130.0, 129.6, 119.6, 117.4, 52.5.

Methyl 3′-cyano-1-(nonyloxy)-[1,1′-biphenyl]-3-carboxylate (4e). The same procedure described for 4c was followed to produce 4e. Under nitrogen atmosphere, a mixture of 3e (52 mg, 0.205 mmol) and K₂CO₃ (57 mg, 0.410 mmol) in anhydrous acetonitrile (5 mL) was stirred at room temperature for 10 min, and then 1-Bromononane (64 mg, 0.308 mmol) was added, and resulting mixture was refluxed for 12 h to obtain 4e as a half white solid (67 mg, 85%). ¹H NMR (400 MHz, CDCl₃) δ 8.00 (d, J=2.2 Hz, 1H), 7.83 (s, 1H), 7.78 (d, J=79 Hz, 1H), 7.67-7.56 (m, 2H), 7.52 (m, J=7.7 Hz, 1H), 7.06 (d, J=8.7 Hz, 1H), 4.08 (t, J=6.5 Hz, 2H), 3.92 (s, 3H), 1.91-1.80 (p, 2H), 1.56-1.43 (p, 2H), 1.42-1.18 (m, 10H), 0.88 (t, J=6.4 Hz, 3H). ¹³C NMR (100 MHz, CDCl₃) δ 166.5, 158.8, 141.0, 131.6, 130.9, 130.5, 130.4, 130.2, 130.1, 129.6, 120.88, 118.8, 69.1, 52.0, 31.8, 29.5, 29.3, 29.2, 29.0, 25.9, 22.6, 14.0.

4-(nonyloxy)-[1,1′-biphenyl]-3,3′-dicarboxylic acid (5e, BPDA3). The same procedure described for the preparation of 5d was followed for 5e. To a solution of 1N NaOH (2 mL) was added a solution of compound 4e (20 mg, 0.052 mmol) in ethanol (2 mL). The resulting mixture was refluxed for 16 h to furnish Se as a white solid (17 mg, 83%). ¹H NMR (400 MHz, methamol-d₄) δ 8.25 (s, 1H), 8.08 (s, 1H), 7.99 (d, J=7.7 Hz, 1H), 7.83 (dd, J=8.5 Hz, 2H), 7.56 (dd, J=7.7 Hz, 1H), 7.24 (d, J=8 7 Hz, 1H), 4.17 (t, J=6.3 Hz, 2H), 1.85 (p, J=6.6 Hz, 2H), 1.52 (p, J=14.6, 7.1 Hz, 2H), 1.34 (m, J=16.9 Hz, 10H), 0.90 (t, J=6.2 Hz, 3H). ¹³C NMR (100 MHz, methamol-d₄) δ 169.7, 159.5, 141.3, 141.3, 133.5, 133.5, 133.2, 133.2, 132.7, 132.0, 132.0, 130.9, 130.9, 130.2, 130.2, 129.4, 128.6, 122.1, 115.0, 115.0, 70.4, 70.4, 33.0, 33.0, 30.7, 30.7, 30.5, 30.4, 30.4, 30.2, 30.2, 27.0, 27.0, 23.7, 14.4. HRMS (ESI) [M−H]⁻: m/z caldt for C₂₃H₂₈O₅: 383.19; found: 383.19.

Methyl3′-(cyanomethyl)-4-hydroxy-[1,1′-biphenyl]-3-carboxylate (3f). Under nitrogen atmosphere, a mixture of 5-Bromo-2-hydroxybenzoic acid methyl ester (1a, 300 mg, 1.29 mmol), (3-(4,4,5,5-Tetramethyl-1,3,2-dioxaborolan-2-yl)benzeneacetonitrile (2f, 662 mg, 2.72 mmol), Pd (PPh₃)₄ (75 mg, 5 mmol %), Cs₂CO₃ (1.26 g, 3.87 mmol) in THF (15 mL) was refluxed for 18 h at 70° C. After the completion of the reaction (as determined by TLC), the mixture was cooled to room temperature, the solvent was evaporated, and the crude residue was dissolved in water (50 mL) and extracted with ethyl acetate (50 mL×3). The extract was washed with water and brine, dried over Na₂SO₄, and evaporated. The residue was purified by column chromatography over silica gel (n-hexane/ethyl acetate=6:4) to furnish 3f as light brown oil (150 mg, 43%). ¹H NMR (400 MHz, CDCl₃) δ 10.81 (s, 1H), 8.06 (d, J=2.4 Hz, 1H), 7.69 (dd, J=8.6, 2.4 Hz, 1H), 7.51 (d, J=9.0 Hz, 2H), 7.44 (dd, J=7.6 Hz, 1H), 7.29 (d, J=7.6 Hz, 1H), 7.08 (d, J=8.7 Hz, 1H), 4.00 (s, 3H), 3.82 (s, 2H). ¹³C NMR (100 MHz, CDCl₃) δ 170.4, 161.3, 141.1, 134.4, 131.5, 130.5, 128.2, 126.5, 126.4, 126.2, 118.3, 112.6, 52.5, 23.7.

Methyl 3′-(cyanomethyl)-4-(nonyloxy)-[1,1′-biphenyl]-3-carboxylate (4f). The same procedure described for the preparation of 4c was followed for 4f. Under nitrogen atmosphere, a mixture of 3f (85 mg, 0.318 mmol), K₂CO₃ (88 mg, 0.636 mmol) in anhydrous acetonitrile 10 mL was stirred at room temperature for 10 min, after which 1-Bromononane (79 mg, 0.382 mmol) was added and the resulting mixture was refluxed for 12 h to give 4f as a colorless oil (100 mg, 82%). ¹H NMR (400 MHz, CDCl₃) δ 8.00 (d, J=2.4 Hz, 1H), 7.65 (dd, J=8.6, 2.4 Hz, 1H), 7.56-7.47 (m, 2H), 7.42 (dt, J=7.6 Hz, 1H), 7.32-7.22 (m, 1H), 7.03 (d, J=8.7 Hz, 1H), 4.07 (t, J=6.5 Hz, 2H), 3.92 (s, 3H), 3.80 (s, 2H), 1.91-1.77 (p, 2H), 1.56-1.45 (p, 2H), 1.42-1.22 (m, 10H), 0.89 (t, J=6.8 Hz, 3H). ¹³C NMR (100 MHz, CDCl₃) δ 166.7, 158.3, 140.8, 132.0, 131.7, 130.4, 130.1, 129.6, 126.4, 126.3, 126.2, 120.7, 117.7, 113.6, 69.0, 52.0, 31.8, 29.5, 29.3, 29.2, 29.0, 25.9, 23.6, 22.6, 14.0.

3′-(carboxymethyl)-4-(nonyloxy)-[1,1′-biphenyl]-3-carboxylic acid (5f, BPDA4). The same procedure described for the preparation of 5d was followed for 5f. To a solution of 1N NaOH (3 mL) was added a solution of compound 4f (38 mg, 0.096 mmol) in ethanol (3 mL), and the resulting mixture was refluxed for 16 h to give 5f as a white solid (35 mg, 92%). ¹H NMR (400 MHz, methanol-d₄) δ 8.05 (s, 1H), 7.77 (d, J=8.7 Hz, 1H), 7.55-7.45 (m, 2H), 7.39 (dt, J=7.6 Hz, 1H), 7.26 (d, J=7.4 Hz, 1H), 7.19 (d, J=8.1 Hz, 1H), 4.14 (t, J=6.2 Hz, 2H), 3.68 (s, 2H), 1.93-1.75 (p, 2H), 1.51 (p, J=14.4, 6.8 Hz, 2H), 1.34 (m, J=21.5, 8.9 Hz, 10H), 0.90 (t, J=6.2 Hz, 3H). ¹³C NMR (100 MHz, methanol-d₄) δ 175.6, 169.8, 159.2, 141.2, 136.8, 134.5, 133.2, 131.0, 130.08, 129.2, 128.7, 126.2, 121.8, 114.9, 70.4, 41.9, 33.0, 30.7, 30.5, 30.4, 30.2, 27.0, 23.7, 15.0. HRMS (ESI) [M+Na]⁺: m/z caldt for C₂₄H₃₀O₅: 421.20; found: 421.20.

Methyl 4′-cyano-4-(nonyloxy)-[1,1′-biphenyl]-2-carboxylate (3g). Under nitrogen atmosphere, 2-Bromo-5-hydroxybenzoic acid methyl ester (1b, 430 mg, 1.86 mmol), 4-(methoxycarbonyl) phenylboronic acid (2g) 704 mg, 3.90 mmol), Pd(PPh₃)₄ (108 mg, 5 mol %), Cs₂CO₃ (1.81 g, 5.58 mmol) in THF (20 mL) was refluxed for 18 h at 70° C. After the completion of the reaction (by TLC), the mixture was cooled to room temperature, the solvent was evaporated, and the crude residue was dissolved in water (100 mL) and extracted with ethyl acetate (50 ml×3). The extract was washed with water and brine, dried over Na₂SO₄, and evaporated. The residue was purified by column chromatography over silica gel (n-hexane/ethyl acetate=7:3) to furnish 3g as a white solid (360 mg, 67%). ¹H NMR (400 MHz, CDCl₃) δ 8.08 (d, J=8.2 Hz, 2H), 7.41 (d, J=2.6 Hz, 1H), 7.37 (d, J=8.1 Hz, 2H), 7.29 (d, J=3.6 Hz, 1H), 7.07 (dd, J=8.4, 2.7 Hz, 1H), 5.86 (s, 1H), 3.97 (s, 3H), 3.65 (s, 3H). ¹³C NMR (100 MHz, CDCl₃) δ 168.6, 167.2, 155.5, 146.0, 134.0, 132.0, 131.5, 130.2, 129.3, 128.5, 127.26, 118.7, 116.9, 52.2.

Methyl 4′-cyano-4-(nonyloxy)-[1,1′-biphenyl]-2-carboxylate (4g). According to the procedure described for the preparation of 4c. the same procedure was followed for 4g. Under nitrogen atmosphere, a mixture of 3g (330 mg, 1.15 mmol), K₂CO₃ (319 mg, 2.30 mmol) in anhydrous acetonitrile 15 mL was stirred at room temperature for 10 min, to that 1-Bromononane (287 mg, 1.38 mmol) was added and resulting mixture was refluxed for 12 h to give 4g as a white solid (450 mg, 94%). ¹H NMR (400 MHz, CDCl₃) δ 8.03 (d, J=8.3 Hz, 2H), 7.35 (d, J=2.7 Hz, 1H), 7.32 (d, J=8.3 Hz, 2H), 7.24 (d, J=1.1 Hz, 1H), 7.05 (dd, J=8.5, 2.7 Hz, 1H), 4.01 (t, J=6.5 Hz, 2H), 3.91 (s, 3H), 3.61 (s, 3H), 1.85-1.74 (p, 2H), 1.46 (p, J=10.4, 4.9 Hz, 2H), 1.40-1.19 (m, 10H), 0.87 (t, J=6.8 Hz, 3H). ¹³C NMR (100 MHz, CDCl₃) δ 168.6, 167.0, 158.6, 146.0, 133.7, 131.7, 131.5, 129.3, 128.5, 118.0, 115.42, 68.4, 52.0, 52.0, 31.8, 29.5, 29.3, 29.2, 29.1, 26.0, 22.6, 14.0.

4-(nonyloxy)-[1,1′-biphenyl]-2,4′-dicarboxylic acid (5g, BPDA5). The same procedure described for the preparation of 5d was followed for making 5g. To a solution of 1N NaOH (5 mL) was added a solution of compound 4g (220 mg, 0.533 mmol) in ethanol (5 mL). The resulting mixture was refluxed for 16 h to give 5g as a light brown solid (189 mg, 92%). ¹H NMR (400 MHz DMSO-D₆) δ 12.91 (s, 1H), 7.93 (d, J=8.0 Hz, 2H), 7.39 (d, J=8.0 Hz, 2H), 7.32 (d, J=8.5 Hz, 1H), 7.26 (d, J=2.5 Hz, 1H), 7.15 (dd, J=8.5. 2.3 Hz, 1H), 4.04 (t, J=6.4 Hz, 2H), 1.81-1.65 (p, 2H), 1.47-1.38 (m, 2H), 1.29 (m, J=25.4 Hz, 10H), 0.86 (t, J=6.5 Hz, 3H). ¹³C NMR (100 MHz, DMSO-D₆) δ 169.5, 167.8, 158.6, 145.7, 133.7, 132.9, 132.3, 129.6, 129.0, 117.7, 115.5, 68.3, 31.8, 29.5, 29.3, 29.2, 29.0, 26.0, 22.6, 14.5. HRMS (ESI) [M−H]⁻: m/z caldt for C₂₃H₂₈O₅: 383.19; found: 383.19.

Methyl 4′-(cyanomethyl)-4-hydroxy-[1,1′-biphenyl]-2-carboxylate (3h). The same procedure described for the preparation of 3g was followed for making 3h. Under nitrogen atmosphere, 2-Bromo-5-hydroxybenzoic acid methyl ester (1b, 500 mg, 2.16 mmol), 4-(4,4,5,5-tetramethyl-1,3,2-dioxaborolan-2-yl)benzeneacetonitrile (2h, 1.10 g, 4.54 mmol), Pd(PPh₃)₄ (125 mg, 5 mol %), Cs₂CO₃ (2.11 g, 6.48 mmol) in THF (20 mL) was refluxed for 18 h at 70° C. to give 3h as a white solid (350 mg, 41%). ¹H NMR (400 MHz, methanol-d₄) δ 7.35 (d, J=8.0 Hz, 2H), 7.25 (d, J=8.1 Hz, 2H), 7.19 (dd, J=11.7, 5.5 Hz, 2H), 6.99 (dd, J=8.4, 2.6 Hz, 1H), 3.91 (s, 3H), 3.61 (s, 3H). ¹³C NMR (100 MHz, methanol-d₄) δ 170.7, 158.1, 142.3, 134.0, 133.1, 133.0, 130.7, 130.2, 128.8, 119.6. 119.47, 117.2, 52.4, 23.2.

Methyl 4′-(cyanomethyl)-4-(nonyloxy)-[1,1′-biphenyl]-2-carboxylate (4h). The same procedure described for the preparation of 4c was followed for making 4h. Under nitrogen atmosphere, a mixture of 3h (110 mg, 0.411 mmol), K₂CO₃ (68 mg, 0.494 mmol) in anhydrous acetonitrile 10 mL was stirred at room temperature for 10 min, to that 1-Bromononane (102 mg, 0.494 mmol) was added and resulting mixture was refluxed for 12 h to furnish 4h as a white solid (130 mg, 80%). ¹H NMR (400 MHz, CDCl₃) δ 7.38 (m, J=10.1, 5.2 Hz, 3H), 7.32 (m, J=8.1 Hz, 2H), 7.29 (d, J=3.2 Hz, 1H), 7.09 (dd, J=8.5, 2.2 Hz, 1H), 4.05 (t, J=6.5 Hz, 2H), 3.82 (s, 3H), 3.69 (s, 3H), 1.90-1.78 (p, 2H), 1.56-1.45 (m, 2H), 1.45-1.25 (m, 10H), 0.92 (t, J=6.5 Hz, 3H). ¹³C NMR (100 MHz, CDCl₃) δ 168.5, 158.4, 141.2, 133.9, 131.9, 131.3, 129.2, 128.3, 127.5, 118.0, 117.8, 115.4, 68.4, 52.0, 31.9, 29.5, 29.3, 29.2, 29.2, 26.0, 23.4, 22.6, 14.0.

4′-(carboxymethyl)-4-(nonyloxy)-[1,1′-biphenyl]-2-carboxylic acid (5h, BPDA6). The procedure described for the preparation of 5d was followed for making 5h. 1N NaOH (3 mL) was added to a solution of compound 4h (80 mg, 0.203 mmol) in ethanol (3 mL). The resulting mixture was refluxed for 16 h to give 5h as a white solid (60 mg, 74%). ¹H NMR (400 MHz, CDCl₃) δ 7.58 (d, J=2.2 Hz, 1H), 7.36-7.26 (m, 5H), 7.16 (dd, J=8.4, 2.3 Hz, 1H), 4.10 (t, J=6.5 Hz, 2H), 3.72 (s, 2H), 1.96-1.82 (p, 2H), 1.54 (m, J=14.2, 6.7 Hz, 2H), 1.40 (m, J=27.5 Hz, 10H), 0.96 (t, J=6.4 Hz, 3H). ¹³C NMR (100 MHz, CDCl₃) δ 178.7, 173.7, 158.2, 140.3, 135.8, 132.0, 131.9, 130.0, 129.0, 128.7, 118.98, 115.9, 68.4, 40.9, 31.9, 29.5, 29.4, 29.2, 29.3, 26.0, 22.7, 14.1. HRMS (ESI) [M+Na]⁺: m/z caldt for C₂₄H₃₀O₅: 421.20, found: 421.20.

Methyl 2′-cyano-4-hydroxy-[1,1′-biphenyl]-3-carboxylate (3i). The same procedure described for the preparation of 3g was followed for making 3i. Under nitrogen atmosphere, 5-Bromo-2-hydroxybenzoic acid methyl ester (1a, 500 mg, 2.16 mmol), 2-cyanophenylboronic acid pinacol ester (2i, 1.040 g, 4.54 mmol), Pd(PPh₃)₄ (125 mg, 5 mol %), Cs₂CO₃ (2.11 g, 6.48 mmol) in THF (20 mL) were mixed and refluxed for 18 h at 70° C. to give 3i as a white solid (250 mg, 45%). ¹H NMR (400 MHz, CDCl₃) δ 10.91 (s, 1H), 8.06 (d, J=2.4 Hz, 1H), 7.75 (d, J=7.8 Hz, 1H), 7.67 (dd, J=8.6, 2.2 Hz, 1H), 7.63 (dd, J=7.7, 0.8 Hz, 1H), 7.49 (d, J=7.8 Hz, 1H), 7.43 (dt, J=7.6 Hz, 1H), 7.11 (d, J=8.6 Hz, 1H), 3.97 (s, 3H). ¹³C NMR (100 MHz, CDCl₃) δ 170.23, 161.91, 144.20, 135.85, 133.71, 132.89, 130.29, 129.80, 129.21, 127.50, 118.56, 118.18, 112.52, 111.10, 52.54.

Methyl 2′-cyano-4-(nonyloxy)-[1,1′-biphenyl]-3-carboxylate (4i). The same procedure described for the preparation of 4c was followed for making 4i. Under nitrogen atmosphere, a mixture of 3i (127 mg, 0.501 mmol), K₂CO₃ (138 mg, 1.00 mmol) in anhydrous acetonitrile 10 mL was stirred at room temperature for 10 min, to that 1-Bromononane (125 mg, 0.602 mmol) was added and resulting mixture was refluxed for 12 h to furnish 4i as a colorless oil (80 mg, 42%). ¹H NMR (400 MHz, CDCl₃) δ 7.96 (d, J=2.3 Hz, 1H), 7.74 (d, J=7.8 Hz, 1H), 7.70 (dd, J=8.6, 2.3 Hz, 1H), 7.62 (dt, J=7.6 Hz, 1H), 7.50 (d, J=7.8 Hz, 1H), 7.41 (dt, J=7.6 Hz, 1H), 7.07 (d, J=8.7 Hz, 1H), 4.13-4.05 (t, 2H), 3.90 (s, 3H), 1.91-1.80 (p, 2H), 1.56-1.44 (m, 2H), 1.38-1.23 (m, 10H), 0.88 (t, J=6.6 Hz, 3H). ¹³C NMR (100 MHz, CDCl₃) δ 166.4, 159.0, 144.2, 133.7, 133.5, 132.8, 132.0, 129.8, 129.7, 127.41, 120.6, 118.6, 113.2, 111.0, 69.0, 52.0, 31.8, 29.5, 29.3, 29.2, 29.0, 25.9, 22.6, 14.0.

4′-(nonyloxy)-[1,1′-biphenyl]-2,3′-dicarboxylic acid (5i, BPDA7). The same procedure described for the preparation of 5d was followed for making 5i; to a solution of 1N NaOH (3 mL) was added a solution of compound 4h (79 mg, 0.205 mmol) in ethanol (3 mL). The resulting mixture was refluxed for 16 h to give 5h as a white solid (65 mg, 81%). ¹H NMR (400 MHz, methanol-d₄) δ 7.92 (d, J=2.4 Hz, 1H), 7.60 (dd, J=8.6, 2.4 Hz, 1H), 7.54 (d, J=7.5 Hz, 1H), 7.51 (dd, J=7.7, 0.9 Hz, 1H), 7.42 (dd, J=7.1, 5.8 Hz, 2H), 7.16 (d, J=8.7 Hz, 1H), 4.15 (t, J=6.4 Hz, 2H), 1.91-1.79 (p, 2H), 1.52 (m, J=14.5, 6.9 Hz, 2H), 1.34 (m, J=13.2 Hz, 10H), 0.91 (t, J=6.5 Hz, 3H). ¹³C NMR (100 MHz, methanol-d₄) δ 174.2, 168.3, 157.9, 138.5, 135.8, 133.7, 132.6, 131.5, 129.9, 129.88, 127.6, 127.0, 120.0, 112.9, 69.0, 31.7, 29.4, 29.3, 29.0, 28.8, 25.7, 22.4, 13.1. HRMS (ESI) [M−H]⁻: m/z caldt for C₂₃H₂₈O₅: 383.19; found: 383.19.

Methyl 2′-cyano-4-hydroxy-[1,1′-biphenyl]-2-carboxylate (3j). The same procedure described for the preparation of 3g was followed for making 3j. Under nitrogen atmosphere, 2-Bromo-5-hydroxybenzoic acid methyl ester (1b, 500 mg, 2.16 mmol), 2-cyanophenylboronic acid pinacol ester (2i, 1.040 g, 4.54 mmol), Pd(PPh₃)₄ (125 mg, 5 mol %), Cs₂CO₃ (2.11 g, 6.48 mmol) in THF (20 mL) were mixed and refluxed for 18 h at 70° C. to give 3j as a brown liquid (220 mg, 40%). ¹H NMR (400 MHz, CDCl₃) δ 7.69 (d, J=7.7 Hz, 1H), 7.58 (m, J=7.7 Hz, 1H), 7.47 (s, 1H), 7.40 (m, J=7.6 Hz, 1H), 7.32 (d, J=7.8 Hz, 1H), 7.15 (d, J=8.3 Hz, 1H), 7.05-6.96 (m, 1H), 6.72 (s, 1H), 3.66 (s, 3H). ¹³C NMR (100 MHz, CDCl₃) δ 167.2, 156.5, 145.9, 132.4, 132.4, 132.3, 131.3, 130.6, 129.9, 127.3, 119.3, 118.4, 117.6, 112.2, 52.3.

Methyl 2′-cyano-4-(nonyloxy)-[1,1′-biphenyl]-2-carboxylate (4j). The same procedure described for the preparation of 4c was followed for making 4j. Under nitrogen atmosphere, a mixture of 3j (130 mg, 0.513 mmol), K₂CO₃ (107 mg, 0.775 mmol) in anhydrous acetonitrile 10 mL was stirred at room temperature for 10 min, to that 1-Bromononane (128 mg, 0.618 mmol) was added and resulting mixture was refluxed for 12 h to give 4j as a colorless oil (110, 56%). ¹H NMR (400 MHz, CDCl₃) δ 7.68 (d, J=7.7 Hz, 1H), 7.62-7.54 (m, 2H), 7.41 (m, J=7.6 Hz, 1H), 7.31 (d, J=7.8 Hz, 1H), 7.21 (d, J=8.5 Hz, 2H), 7.11 (dd, J=8.5, 2.3 Hz, 1H), 4.03 (t, J=6.5 Hz, 2H), 3.73-3.63 (s, 3H), 1.89-1.75 (q, 2H), 1.46 (m, J=14.0, 6.3 Hz, 2H), 1.27 (m, J=16.2 Hz, 10H), 0.88 (t, J=9.0, 4.0 Hz, 3H). ¹³C NMR (100 MHz, CDCl₃) δ 166.9, 159.2, 145.8, 132.3, 132.2, 132.0, 131.6, 130.7, 129.8, 127.2, 118.5, 118.3, 116.2, 112.6, 68.4, 52.1, 31.9, 29.5, 29.4, 29.3, 29.2, 26.0, 22.66, 14.1.

4-(nonyloxy)-[1,1′-biphenyl]-2,2′-dicarboxylic acid (5j, BPDA8). The same procedure described for the preparation of 5d was followed for making 5j. To a solution of 1N NaOH (4 mL) was added a solution of compound 4j (110 mg, 0.290 mmol) in ethanol (4 mL). The resulting mixture was refluxed for 16 h to give 5j as a white solid (90 mg, 81%). ¹H NMR (400 MHz, DMSO-D₆) δ 7.55-7.47 (m, 1H), 7.45-7.36 (m, 2H), 7.36-7.22 (m, 2H), 7.10-7.04 (m, 2H), 4.02 (t, J=6.4 Hz, 2H), 1.82-1.67 (m, 2H), 1.43 (m, 2H), 1.27 (m, 10 H), 0.86 (t, J=6.2 Hz, 3H). ¹³C NMR (100 MHz, DMSO-D₆) δ 171.4, 169.1, 158.1, 139.6, 136.5, 133.5, 133.2, 132.0, 130.8, 130.6, 130.0, 129.5, 127.6, 127.4, 117.2, 114.8, 68.2, 31.8, 29.5, 29.3, 29.2, 26.0, 22.6, 14.4. HRMS (ESI) [M−H]⁻: m/z caldt for C₂₃H₂₈O₅: 383.19; found: 383.19.

Methyl 3′-cyano-4-hydroxy-[1,1′-biphenyl]-2-carboxylate (3k). The same procedure described for the preparation of 3g was followed for making 3k. Under nitrogen atmosphere, 2-Bromo-5-hydroxybenzoic acid methyl ester (1b, 500 mg, 2.16 mmol), 3-cyanophenylboronic acid pinacol ester (2e, 595 g, 2.59 mmol), Pd(PPh₃)₄ (35 mg, 5 mol %), Cs₂CO₃ (2.11 g, 6.48 mmol) in THF (20 mL) were mixed and refluxed for 18 h at 70° C. to give 3k as a white solid (267 mg, 48%). ¹H NMR (400 MHz, CDCl₃) δ 7.59 (d, J=7.0 Hz, 1H), 7.54 (s, 1H), 7.46 (m, J=8.2 Hz, 2H), 7.39 (d, J=2.4 Hz, 1H), 7.14 (d, J=8.4 Hz, 1H), 7.02 (dd, J=8.4, 2.4 Hz, 1H), 3.65 (s, 3H). ¹³C NMR (100 MHz, CDCl₃) δ 168.2, 156.1, 142.5, 133.2, 132.5, 132.0, 132.0, 130.8, 130.4, 128.7, 119.0, 118.8, 117.2, 111.8, 52.2.

Methyl 3′-cyano-4-(nonyloxy)-[1,1′-biphenyl]-2-carboxylate (4k). The same procedure described for the preparation of 4c was followed for preparation of 4k. Under nitrogen atmosphere, a mixture of 3k (267 mg, 10 mmol), K₂CO₃ (219 mg, 1.58 mmol) in anhydrous acetonitrile 10 mL was stirred at room temperature for 10 min, to that 1-Bromononane (262 mg, 1.26 mmol) was added and resulting mixture was refluxed for 12 h to give 4k as a white solid (256 mg, 64%). ¹H NMR (400 MHz, CDCl₃) δ 7.59 (dd, J=6.8, 1.6 Hz, 1H), 7.54 (s, 1H), 7.47 (dd, J=11.3, 4.7 Hz, 2H), 7.41 (d, J=3.1 Hz, 1H), 7.19 (d, J=8.5 Hz, 1H), 7.06 (dd, J=8.5, 2.6 Hz, 1H), 4.01 (t, J=6.5 Hz, 2H), 3.65 (s, 3H), 1.86-1.74 (q, 2H), 1.53-1.40 (m, 2H), 1.40-1.22 (m, 10H), 0.87 (t, J=6.7 Hz, 3H). ¹³C NMR (100 MHz, CDCl₃) δ 167.8, 158.8, 142.6, 133.0, 132.7, 132.0, 131.8, 131.0, 130.4, 128.6, 118.8, 118.2, 115.8, 112.0, 68.4, 52.0, 31.8, 29.5, 29.3, 29.2, 29.1, 26.0, 22.6, 14.0.

4-(nonyloxy)-[1,1′-biphenyl]-2,3′-dicarboxylic acid (5k, BPDA9). The same procedure described for the preparation of 5d was followed for making 5k. To a solution of 1N NaOH (5 mL) was added a solution of compound 4j (256 mg, 1.37 mmol) in ethanol (5 mL). The resulting mixture was refluxed for 16 h to give 5k as a light brown solid (230 mg, 88%). ¹H NMR (400 MHz, DMSO-D₆) δ 7.89 (d, J=7.0 Hz, 1H), 7.84 (s, 1H), 7.51 (m, J=7.7 Hz, 2H), 7.31 (d, J=8.5 Hz, 1H), 7.26 (d, J=2.5 Hz, 1H), 7.14 (dd, J=8.5, 2.6 Hz, 1H), 4.03 (t, J=6.4 Hz, 3H), 1.72 (q, J=14.2, 6.8 Hz, 2H), 1.48-1.37 (m, 2H), 1.37-1.19 (m, 10H), 0.85 (t, J=6.6 Hz, 3H). ¹³C NMR (100 MHz, DMSO-D₆) δ 169.6, 167.8, 158.4, 141.6, 133.6, 133.2, 132.9, 132.3. 131.0, 129.7, 128.9, 128.1, 117.7, 115.4, 68.3, 31.8, 29.5, 29.6, 29.2, 29.0, 26.0, 22.6, 14.4 HRMS (ESI) [M−H]⁻: m/z caldt for C₂₃H₂₈O₅: 383.19; found: 383.19.

Methyl 3′-(cyanomethyl)-4-hydroxy-[1,1′-biphenyl]-2-carboxylate (3l). The same procedure described for the preparation of 3g was followed for making 3l. Under nitrogen atmosphere, 2-Bromo-5-hydroxybenzoic acid methyl ester (1b, 500 mg, 2.16 mmol), 3-cyanophenylboronic acid pinacol ester (2f, 788 mg, 3.24 mmol), Pd(PPh₃)₄ (35 mg, 5 mol %), Cs₂CO₃ (2.11 g, 6.48 mmol) in THF (20 mL) were mixed and refluxed for 18 h at 70° C. to give 3l as a white solid (220 mg, 38%). ¹H NMR (400 MHz, CDCl₃) δ 7.37 (dd, J=13.6, 6.0 Hz, 1H), 7.32 (d, J=2.6 Hz, 2H), 7.29-7.17 (m, 4H), 7.00 (dd, J=8.4, 2.6 Hz, 1H), 5.82 (d, J=13.0 Hz, 1H), 3.77 (s, 2H), 3.65 (s, 3H). ¹³C NMR (100 MHz, CDCl₃) δ 168.6, 155.3, 142.1, 134.0, 132.2, 131.4, 129.6, 128.7, 128.31, 128.0, 126.4, 118.7, 117.9, 116.8, 52.2, 23.6.

Methyl 3′-(cyanomethyl)-4-(nonyloxy)-[1,1′-biphenyl]-2-carboxylate (4l). The same procedure described for the preparation of 4c was followed for preparation of 4l. Under nitrogen atmosphere, a mixture of 3l (180 mg, 0.674 mmol), K₂CO₃ (140 mg, 1.0 mmol) in anhydrous acetonitrile 10 mL was stirred at room temperature for 10 min, to that 1-Bromononane (168 mg, 0.808 mmol) was added and resulting mixture was refluxed for 12 h to give 4l as a colorless oil (200 mg, 75%). ¹H NMR (400 MHz, CDCl₃) δ 7.36 (dd, J=9.3, 5.1 Hz, 2H), 7.32-7.18 (m, 4H), 7.05 (dd, J=8.4, 2.5 Hz, 1H), 4.01 (t, J=6.4 Hz, 2H), 3.76 (s, 2H), 3.65 (s, 3H), 1.89-1.72 (q, 2H), 1.54-1.41 (m, 2H), 1.31 (m, J=21.9 Hz, 10H), 0.87 (t, J=6.8 Hz, 3H). ¹³C NMR (100 MHz, CDCl₃) δ 168.5, 158.4, 142.2, 133.8, 131.8, 131.4, 129.6, 128.7, 128.3, 128.0, 126.3, 118.0, 117.8, 115.4, 68.3, 52.0, 31.8, 29.5, 29.3, 29.2, 29.1, 26.0, 23.6, 22.6, 14.0.

3′-(carboxymethyl)-4-(nonyloxy)-[1,1′-biphenyl]-2-carboxylic acid (5l, BPDA10). The same procedure described for the preparation of 5d was followed for making 5l. To a solution of 1N NaOH (5 mL) was added a solution of compound 4l (130 mg, 0.330 mmol) in ethanol (5 mL). The resulting mixture was refluxed for 16 h to give 5l as a white solid (100 mg, 76%). ¹H NMR (400 MHz, CDCl₃) δ 7.52 (d, J=2.5 Hz, 1H), 7.33 (m, J=7.7 Hz, 1H), 7.23-7.18 (m, 2H), 7.16 (d, J=7.4 Hz, 2H), 7.07 (dd, J=8.5, 2.6 Hz, 1H), 4.01 (t, J=6.5 Hz, 2H), 3.60 (s, 2H), 1.86-1.74 (q, 2H), 1.45 (dd, J=14.8, 7.1 Hz, 2H), 1.32 (m, J=27.2 Hz, 10H), 0.88 (t, J=6.6 Hz, 3H). ¹³C NMR (100 MHz, CDCl₃) δ 178.7, 173.6, 158.2, 141.3, 135.8, 132.3, 132.3, 131.2, 129.6, 128.5, 128.0, 127.0, 119.1, 116.2, 68.4, 41.3, 31.9, 29.5, 29.4, 29.3, 29.2, 26.0, 22.7, 14.1. HRMS (ESI) [M+Na]⁺: m/z caldt for C₂₄H₃₀O₅: 421.20; found: 421.20.

PTPase Assay. The inhibitory effect of the ten compounds on the enzyme activity of SHP2, SHP1, and PTP1B was determined by the PTPase assay as described previously [39, 40]. Production and purification of the GST fusions of the PTP domains of SHP2, SHP1, and PTP1B were performed as reported by us recently [41]. DiFMUP (6,8-Difluoro-4-Methylumbelliferyl Phosphate) was purchased from ThermoFisher scientific. Briefly, competent E. coli were transformed with the GST fusion plasmids, and proteins were expressed using a standard protocol, purified by a glutathione conjugated sepharose column, and quantified by spectroscopic measurement of absorbance at 280 nm wavelength. Proteins were eluted into a phosphatase buffer containing 10 mM Tri-HCl, 100 mM NaCl, 1 mM EDTA, and 0.01% Tween-20, pH7.2. For the PTPase reactions, purified PTP domains of SHP2, SHP1 or PTP1B were added first (1 nM), followed by the inhibitors in serial dilutions (12 nM-100 μM). The reactions were started by adding the artificial substrate difluoromethylumbelliferyl phosphate (DiFMUP) to a final concentration of 20 μM for SHP2, 35 μM for SHP1, and 10 μM for PTP1B in a reaction volume of 100 μl. The different concentrations of DiFMUP used in the reactions were based the reported Km values for each PTP [42]. After incubation at 37° C. for 20 minutes, fluorescence intensity of the reactions was measured by Synergy 4 plate reader Graphpad Prism was used to calculate the IC₅₀ values.

Cells, Cell Culture, and Reagents. We have used the HER2-positive JIMT-1 and the triple-negative MDA-MB468 breast cancer cell lines for determining the anti-breast cancer cell effect of the selected compounds. While the MDA-MB468 cells were purchased from the American Tissue Culture Collection (ATCC), the JIMT-1 cells were purchased from DSMZ, Germany Both cells were grown in Dulbecco's modified Eagle's medium (DMEM) supplemented with 10% fetal bovine serum. DiFMUP was from Invitrogen, glutathione-sepharose beads were from GE Healthcare, anti-HER2, anti-SHP2, and anti-panERK2 antibodies were from BD biosciences, anti-β-actin antibody was from Sigma-Aldrich, and anti-phospho-ERK1/2, anti-phospho-Akt, anti-panAkt, anti-EGFR, anti-HER2, anti-MET, and anti-FGFR antibodies were from Cell Signaling.

Cellular thermal shift assay (CETSA). The CETSA assay is based on the principle that drug binding to a target protein confers stability from heat-induced unfolding and precipitation when compared to the non-drug bound counterpart [35]. For the CETSA assay, cells were grown to approximately 80% density and lysed with a lysis buffer described for the IB analyses below. The pan-PTP inhibitor orthovanadate was removed from the lysis buffer to avoid interference with binding of BPDA2 to SHP2 Total cell lysates (TCL) prepared from two breast cancer cell lines, namely the HER2-positive JIMT-1 and the triple-negative MDA-MB-468, were cleared by centrifugation, mixed with 100 μM BPDA2, incubated at room temperature for 10 minutes to allow binding, divided into 10 aliquots, and then treated with heat, ranging from 37° C. to 64° C. that differ by 3° C. intervals, for 10 minutes. After heat treatment, samples were centrifuged at 20,000 g for 20 minutes at 4° C. to remove sediments of the precipitated proteins, supernatants mixed with Laemmli sample buffer, and analyzed by IB for SHP2 and multiple other non-target proteins. Non-BPDA2 treated samples were used as controls.

Immunoblotting (IB) analyses. For evaluating effect of BPDA2 on SHP2 basal signaling, cells were grown to approximately 90% density and lysed in a buffer containing 20 mM Tris-HCl (pH 7.4), 150 mM NaCl, 1 mM EDTA, 1% Triton-X-100, 10% glycerol, and 50 mM NaF supplemented with a cocktail of protease inhibitors and 1 mM sodium orthovanadate. Lysates containing comparable protein levels were denatured by adding equal volume of 2× Laemmli sample buffer and boiling at 100° C. for 5 minutes. The proteins were separated by polyacrylamide (8%) gel electrophoresis (PAGE), immobilized onto a nitrocellulose membrane, blocked with 3% bovine serum albumin (BSA) in tris-buffered saline containing 1% Tween-20 (TBST), and stained with primary antibodies for 2 hours at room temperature or overnight at 4° C. as desired. We followed a standard protocol for secondary antibody staining, signal detection, and visualization by chemiluminescence method (Pierce Inc.).

Anchorage-Independent Growth Assay. We used the anchorage-independent growth assay, also known as the soft agar colony formation assay, to determine effect of BPDA2 on the transformation phenotype of breast cancer cells assay as described previously [3, 4]. Initially, a stock solution of 5% agar was prepared by mixing powdered agar with PBS (W/V) and boiling in a microwave. Next, the liquefied agar at approximately 45° C. was added to cell growth medium at 0.3%, and used for overlaying 6 cm cell culture plates (˜2 ml/plate). Next, cells suspended in 2 ml of growth medium were mixed with melted agar to a final concentration of 0.3% and immediately poured onto the agar overlay. BPDA2 was added to cells at variable concentrations (0.5-4 μM) and cultured for approximately 10 days with additional feeding every 3 days. Colony formation was visualized under a microscope after staining with crystal violet, and images were collected using an Olympus IX71 microscope equipped with DP80 color camera controlled by a cellSens software.

Mammosphere Formation Assay. We used the mammosphere or tumorisphere assay to determine the effect of BPDA2 on cancer stem cell (CSC) properties of the JIMT-1 and the MDA-MB-468 breast cancer cells, following the protocol described by us and others previously [1, 43]. Briefly, approximately 10⁶ cells were cultured in serum-free DMEM containing 1 μg/ml hydrocortisone, 10 μg/ml insulin, 10 ng/ml EGF, 10 ng/ml FGF, 5 ng/ml heparin, and B27 (Invitrogen) in 6 cm ultra-low adherence culture plates. Cells were treated with vehicle or BPDA2 at various concentrations (0.5-4 μM) continuously for 10 days. At the end, spheres were collected in smaller volume of media using centrifugation, and imaged using the 10× objective in an Olympus IX71 microscope equipped with DP80 color camera controlled by a cellSens software. Images that represent each experimental group are presented for visual appreciation of differences in size and number. Also, we have estimated the observed differences by counting all viable spheres in three 10× objective fields in each case.

In certain embodiments of this invention the following are provided:
1. A compound of the formula that is one selected from the group consisting of BPDA1, BPDA2, BPDA3, BPDA4, BPDA5, BPDA6, BPDA7, BPDA8, BPDA9, and BPDA10, as set forth in Table 1.
2. A compound of the formula BPDA1, BPDA2, BPDA3, BPDA4, BPDA5, BPDA6, BPDA7, BPDA8, BPDA9, or BPDA10 that is a phosphotyrosyl phosphatase 2 inhibitor.
3. A pharmaceutical composition comprising a compound having the formula of one selected from the group consisting of BPDA1, BPDA2, BPDA3, BPDA4, BPDA5, BPDA6, BPDA7, BPDA8, BPDA9, and BPDA10, and an acceptable pharmaceutical carrier.
4. A method of treating a patient having cancer comprising administering a therapeutically effective amount of a compound of the formula BPDA1, BPDA2, BPDA3, BPDA4, BPDA5, BPDA6, BPDA7, BPDA8, BPDA9, or BPDA10.
5. A method of treating a patient having cancer comprising administering a therapeutically effective amount of a pharmaceutical composition comprising a compound that is one selected from the group consisting of formula BPDA1, BPDA2, BPDA3, BPDA4, BPDA5, BPDA6, BPDA7, BPDA8, BPDA9, and BPDA10, and an acceptable pharmaceutical carrier.
6. A compound that is a derivative of CNBDA.
7. A compound that is a derivative of CNBDA that is a phosphotyrosyl phosphatase 2 inhibitor.
8. The compound of item 7 above that is one selected from the group consisting of the formula BPDA1, BPDA2, BPDA3, BPDA4, BPDA5, BPDA6, BPDA7, BPDA8, BPDA9, and BPDA10.
9. A compound of the formula 4′-(carboxymethyl)-4-(nonyloxy)-[1,1′-biphenyl]-3-carboxylic acid (BPDA2).

As used herein, the term “patient” means members of the animal kingdom, including, but not limited to human beings.

As used herein, the term “effective amount” or “therapeutically effective amount” refers to that amount of the present compounds, and salts thereof, and/or compositions required to bring about a desired effect in a patient. The desired effect will vary depending upon the illness or disease state being treated. For example, the desired effect may be reducing the tumor size, destroying cancerous cells, and/or preventing metastasis, and one of which may be the desired therapeutic response. On its most basic level, a therapeutically effective amount is that amount of a substance that is needed to inhibit mitosis of a cancerous cell.

As used herein, an “acceptable pharmaceutical carrier” refers to any pharmaceutical carrier known in the art, absent compatibility problems with the novel compounds of this invention. Generally, the pharmaceutical carrier includes, for example, but not limited to, physiologic saline, 5% dextrose in water, combinations thereof, lactose, sucrose, gelatin, and polymers.

It is well within the skill of one practicing in the art, a therapeutically effective amount of the compounds of this invention may be administered by any means known in the art, including but not limited to, injection, parenterally, intravenously, intraperitoneally, orally, rectally, and topically.

The compounds of this invention or pharmaceutically acceptable salts or hydrates of the compounds of this invention, can be incorporated into, for example but not limited to, tablets, capsules, elixirs, suspensions, solutions, syrups, and sustained-release preparations and formulations. The compounds of this invention may be incorporated with, for example but not limited to, binders, excipients, disintegrating agents, lubricants, sweetening agents, preservatives, dyes, and flavoring agents.

### REFERENCES—SECTION I

1. Matalkah F. Martin E, Zhao H, Agazie Y M: SHP2 acts both upstream and downstream of multiple receptor tyrosine kinases to promote basal-like and triple-negative breast cancer. Breast cancer research: BCR 2016, 18.2.
2. Zhou X, Coad J, Ducatman B, Agazie Y M: SHP2 is up-regulated in breast cancer cells and in infiltrating ductal carcinoma of the breast, implying its involvement in breast oncogenesis. Histopathology 2008, 53:389-402.
3. Zhou X D, Agazie Y M: Inhibition of SHP2 leads to mesenchymal to epithelial transition in breast cancer cells. Cell death and differentiation 2008, 15:988-996.
4. Zhou X, Agazie Y M: Molecular mechanism for SHP2 in promoting HER2-induced signaling and transformation. The Journal of biological chemistry 2009, 284: 12226-12234.
5. Hartman Z R, Schaller M D, Agazie Y M: The tyrosine phosphatase SHP2 regulates focal adhesion kinase to promote EGF-induced lamellipodia persistence and cell migration. Molecular cancer research: MCR 2013, 11:651-664.
6. Zhao H, Agazie Y M: Inhibition of SHP2 in basal-like and triple-negative breast cells induces basal-to-luminal transition, hormone dependency, and sensitivity to anti-hormone treatment. BMC cancer 2015, 15:109.
7. Aceto N, Sausgruber N, Brinkhaus H, Gaidatzis D, Martiny-Baron G, Mazzarol G, Confalonieri S, Quarto M, Hu G, Balwierz P J, et al: Tyrosine phosphatase SHP2 promotes breast cancer progression and maintains tumor-initiating cells via activation of key transcription factors and a positive feedback signaling loop. Nature medicine 2012, 18:529-537.
8. Agazie Y. Ischenko I, Hayman M: Concomitant activation of the PI3K-Akt and the Ras-ERK signaling pathways is essential for transformation by the V-SEA tyrosine kinase oncogene. Oncogene 2002, 21:697-707.
9. Agazie Y M, Hayman M J: Molecular mechanism for a role of SHP2 in epidermal growth factor receptor signaling. Molecular and cellular biology 2003, 23:7875-7886.
10. Agazie Y M, Movilla N, Ischenko I, Hayman M J: The phosphotyrosine phosphatase SHP2 is a critical mediator of transformation induced by the oncogenic fibroblast growth factor receptor 3. Oncogene 2003, 22:6909-6918.
11. Burks J, Agazie Y M: Modulation of alpha-catenin Tyr phosphorylation by SHP2 positively effects cell transformation induced by the constitutively active FGFR3. Oncogene 2006, 25:7166-7179.
12. Li J, Reed S A, Johnson S E: Hepatocyte growth factor (HGF) signals through SHP2 to regulate primary mouse myoblast proliferation. Experimental cell research 2009, 315:2284-2292.
13. Miura K, Wakayama Y, Tanino M, Orba Y, Sawa H, Hatakeyama M, Tanaka S, Sabe H, Mochizuki N: Involvement of EphA2-mediated tyrosine phosphorylation of Shp2 in Shp2-regulated activation of extracellular signal-regulated kinase. Oncogene 2013.
14. Zhang S Q, Yang W, Kontaridis M I, Bivona T G, Wen G, Araki T, Luo J, Thompson J A, Schraven B L, Philips M R, Neel B G: Shp2 regulates SRC family kinase activity and Ras/Erk activation by controlling Csk recruitment. Molecular cell 2004, 13:341-355.
15. Feng G S, Hui C C, Pawson T: SH2-containing phosphotyrosine phosphatase as a target of protein-tyrosine kinases. Science 1993, 259:1607-1611.
16. Feng G S, Shen R, Heng H H, Tsui L C, Kazlauskas A, Pawson T: Receptor-binding, tyrosine phosphorylation and chromosome localization of the mouse SH2-containing phosphotyrosine phosphatase Syp. Oncogene 1994, 9:1545-1550.
17. Hof P, Pluskey S, Dhe-Paganon S, Eck M J, Shoelson S E: Crystal structure of the tyrosine phosphatase SHP-2. Cell 1998, 92:441-450.
18. Zhou X D, Agazie Y M: Molecular Mechanism for SHP2 in Promoting HER2-induced Signaling and Transformation. Journal of Biological Chemistry 2009, 284:12226-12234.
19. Wilken J A, Maihle N J: Primary trastuzumab resistance: new tricks for an old drug. Annals of the New York Academy of Sciences, 1210:53-65.
20. Zhuang G, Brantley-Sieders D M, Vaught D, Yu J, Xie L, Wells S, Jackson D, Muraoka-Cook R, Arteaga C, Chen J: Elevation of receptor tyrosine kinase EphA2 mediates resistance to trastuzumab therapy. Cancer research, 70:299-308.
21. Qiu W, Wang X, Romanov V, Hutchinson A, Lin A, Ruzanov M, Battaile K P, Pai E F, Neel B G, Chirgadze N Y: Structural insights into Noonan/LEOPARD syndrome-related mutants of protein-tyrosine phosphatase SHP2 (PTPN11). BMC structural biology 2014, 14:10.
22. Bonetti M, Paardekooper Overman J, Tessadori F, Noel E, Bakkers J, den Hertog J: Noonan and LEOPARD syndrome Shp2 variants induce heart displacement defects in zebrafish. Development 2014, 141:1961-1970.
23. Hellmuth K, Grosskopf S, Lum C T, Wurtele M, Roder N, von Kries J P, Rosario M, Rademann J, Birchmeier W. Specific inhibitors of the protein tyrosine phosphatase Shp2 identified by high-throughput docking. Proceedings of the National Academy of Sciences of the United States of America 2008, 105:7275-7280.
24. Chen L, Pernazza D, Scott L M, Lawrence H R, Ren Y, Luo Y, Wu X, Sung S S, Guida W C, Sebti S M, et al: Inhibition of cellular Shp2 activity by a methyl ester analog of SPI-112. Biochemical pharmacology 2010, 80:801-810.
25. Zhang X, He Y, Liu S, Yu Z, Jiang Z X, Yang Z, Dong Y, Nabinger S C, Wu L, Gunawan A M, et al: Salicylic acid based small molecule inhibitor for the oncogenic Src homology-2 domain containing protein tyrosine phosphatase-2 (SHP2). Journal of medicinal chemistry 2010, 53:2482-2493.
26. Zeng L F, Zhang R Y, Yu Z H, Li S, Wu L, Gunawan A M, Lane B S, Mali R S, Li X, Chan R J, et al: Therapeutic potential of targeting the oncogenic SHP2 phosphatase. Journal of medicinal chemistry 2014, 57:6594-6609.
27. Yu B, Liu W, Yu W M, Loh M L, Alter S, Guvench O, Mackerell A D, Jr., Tang L D, Qu C K: Targeting Protein Tyrosine Phosphatase SHP2 for the Treatment of PTPN11-Associated Malignancies. Molecular cancer therapeutics 2013.
28. Mostinski Y, Heynen G J J E, Lopez-Alberca M P, Paul J, Miksche S, Radetzki S, Schaller D, Shanina E, Seyffarth C, Kolomeets Y, et al: From Pyrazolones to Azaindoles: Evolution of Active-Site SHP2 Inhibitors Based on Scaffold Hopping and Bioisosteric Replacement. Journal of medicinal chemistry 2020, 63:14780-14804.
29. Hartman Z, Geldenhuys W J, Agazie Y M: Novel Small-Molecule Inhibitor for the Oncogenic Tyrosine Phosphatase SHP2 with Anti-Breast Cancer Cell Effects. Acs Omega 2020, 5:25113-25124.
30. Chen Y N, LaMarche M J, Chan H M, Fekkes P, Garcia-Fortanet J, Acker M G, Antonakos B, Chen C H, Chen Z, Cooke V G, et al: Allosteric inhibition of SHP2 phosphatase inhibits cancers driven by receptor tyrosine kinases. Nature 2016, 535:148-152.
31. Garcia Fortanet J, Chen C H, Chen Y N, Chen Z, Deng Z, Firestone B, Fekkes P, Fodor M, Fortin P D, Fridrich C, et al: Allosteric Inhibition of SHP2: Identification of a Potent, Selective, and Orally Efficacious Phosphatase Inhibitor. Journal of medicinal chemistry 2016, 59:7773-7782.
32. Czako B, Sun Y, McAfoos T, Cross J B, Leonard P G, Burke J P, Carroll C L, Feng N, Harris A L, Jiang Y, et al: Discovery of 6-[(3S,4S)-4-Amino-3-methyl-2-oxa-8-azaspiro[4.5]decan-8-yl]-3-(2,3-dichlorophenyl)-2-methyl-3,4-dihydropyrimidin-4-one (IACS-15414), a Potent and Orally Bioavailable SHP2 Inhibitor. Journal of medicinal chemistry 2021.
33. Halgren T A, Murphy R B, Friesner R A, Beard H S, Frye L L, Pollard W T, Banks J L: Glide: A new approach for rapid, accurate docking and scoring. 2. Enrichment factors in database screening. Journal of medicinal chemistry 2004, 47:1750-1759.
34. Yu Z H, Xu J, Walls C D, Chen L, Zhang S, Zhang R, Wu L, Wang L, Liu S, Zhang Z Y: Structural and mechanistic insights into LEOPARD syndrome-associated SHP2 mutations. The Journal of biological chemistry 2013, 288:10472-10482.
35. Al-Amin R A, Gallant C J, Muthelo P M, Landegren U: Sensitive Measurement of Drug-Target Engagement by a Cellular Thermal Shift Assay with Multiplex Proximity Extension Readout. Anal Chem 2021, 93:10999-11009.
36. Zhao H, Martin E, Matalkah F, Shah N, Ivanov A, Ruppert J M, Lockman P R, Agazie Y M: Conditional knockout of SHP2 in ErbB2 transgenic mice or inhibition in HER2-amplified breast cancer cell lines blocks oncogene expression and tumorigenesis. Oncogene 2019, 38:2275-2290.
37. Pawar A B, Lade D M: Cobalt(III)-catalyzed C—H halogenation of 6-arylpurines: facile entry into arylated, sulfenylated and alkoxylated 6-arylpurines. Org Biomol Chem 2016, 14:3275-3283.
38. Yang X F, Zhu H B, Liu M: Transition-metal-based (Co2+, Ni2+ and Cd2+) coordination polymers constructed by a polytopic ligand integrating both flexible aliphatic and rigid aromatic carboxylate groups: Aqueous detection of nitroaromatics. Polyhedron 2017, 128:18-29.
39. Krishnan N, Bencze G, Cohen P, Tonks N K: The anti-inflammatory compound BAY-11-7082 is a potent inhibitor of protein tyrosine phosphatases. The FEBS journal 2013, 280-2830-2841.
40. Lopez S M, Hodgson M C, Packianathan C, Bingol-Ozakpinar O, Uras F, Rosen B P, Agoulnik T U: Determinants of the tumor suppressor INPP4B protein and lipid phosphatase activities. Biochemical and biophysical research communications 2013, 440:277-282.
41. Hartman Z, Geldenhuys W J, Agazie Y M: A specific amino acid context in EGFR and HER2 phosphorylation sites enables selective binding to the active site of Src homology phosphatase 2 (SHP2). The Journal of biological chemistry 2020.
42. Chen L, Sung S S, Yip M L, Lawrence H R, Ren Y, Guida W C, Sebti S M, Lawrence N J, Wu J: Discovery of a novel shp2 protein tyrosine phosphatase inhibitor. Molecular pharmacology 2006, 70:562-570.
43. Dontu G, Abdallah W M, Foley J M, Jackson K W, Clarke M F, Kawamura M J, Wicha M S: In vitro propagation and transcriptional profiling of human mammary stem/progenitor cells. Genes & development 2003, 17:1253-1270.

### Experimental Section II

The Src homology phosphotyrosyl phosphatase 2 (SHP2) is an oncogenic protein for which targeted therapies are being sought. In line with this idea, we have previously reported the development of a specific active site inhibitor named CNBDA that showed effectivity in suppressing the transformation phenotypes of breast cancer cells. To improve efficacy, we introduced limited modifications to the parent compound and tested potency in vitro and under cell culture conditions. Of these modifications, removal of one of the butyric acid groups led to the production of a compound named CNBCA, which showed a 5.7-fold better potency against the SHP2 enzyme activity in vitro. In addition, CNBCA showed better selectivity to SHP2 than the control PTPs (SHP1 and PTP1B) as determined by the phosphatase assay. Furthermore, CNBCA binds and inhibits enzyme activity of full-length SHP2 in cellular contexts, downregulates SHP2 mediated signaling, and suppresses breast cancer cell phenotypes, including cell proliferation, colony formation, and mammosphere growth. These findings show that targeting SHP2 with CNBCA is effective against the cancerous properties of breast cancer cells. FIG. 5 shows the structures of the compounds CNBCA and CNBBA of this invention.

The Src homology phosphotyrosyl phosphatase 2 (SHP2) is a positive regulator of receptor tyrosine kinase (RTK) signaling,¹⁻⁶ and by doing so it promotes the cancerous phenotype of tumor cells, including breast cancer.^{7,8} For instance, SHP2 promotes the transformation phenotypes of the HER2-positive (HER2+) and the triple-negative breast cancer (TNBC) cells, including epithelial to mesenchymal transition in 2D culture, colony formation in soft agar, mammosphere formation in suspension cultures, extracellular matrix degradation and invasion in 3D matrigel, and tumorigenesis in vivo.⁷⁻¹¹

SHP2 is a 595 amino acid long cytoplasmic phosphotyrosyl phosphatase (PTP) encoded by the PTPN11 gene (NCBI accession ID: XP_054228694.1). The SHP2 protein is composed of two SH2 domains in the N-terminal and a phosphotyrosyl phosphatase (PTP) domain in the C-terminal regions.¹² The SH2 domains play an adaptor role by mediating interaction with phosphotyrosine (pTyr) on signaling proteins that function in the RTK signaling pathway. This allows the PTP domain to access tyrosine phosphorylated biological substrates and catalyze dephosphorylation reactions.^{13,14} How a tyrosine phosphatase becomes a requisite signal transducer in a signaling pathway that depends on tyrosine phosphorylation has not been fully understood. Available literature suggests that SHP2 specifically dephosphorylates negative regulatory pTyr sites in RTKs and downstream signaling proteins to augment and sustain downstream signaling. For instance, SHP2 dephosphorylates RasGAP (Ras GTPase) docking sites on EGFR (pTyr992) and on HER2 (pTyr1023) to sustain the activated form of Ras (GTP-Ras).^{2,15} SHP2 has also been shown to dephosphorylate pTyr397 in FAK to facilitate focal adhesion turnover¹⁶ and pTyr314 in PAG to enhance Src Tyr kinase signaling.¹⁷ The positive role of SHP2 in tyrosine kinase signaling in general has attracted interest to develop specific inhibitors for use in diseases, particularly in cancer, that often involve hyperactive tyrosine kinase signaling. The oncogenic property of SHP2 has spurred much interest for developing targeted therapies in cancer. As such, significant efforts have been devoted to developing specific inhibitors. Initial efforts primarily focused on developing active-site SHP2 inhibitors (ASSIs) that competitively inhibit PTPase (enzyme) activity. Notable active site inhibitors that have been reported and used by other investigators for inhibiting SHP2 include: PHPS1,¹⁸ SPI-112Me,¹⁹ II-B08,²⁰ 11a-1,²¹ 220-32,²² compound 45,²³ and our compound CNBDA.²⁴ Relatively recently, inhibitors that bind to allosteric clefts in SHP2 have been reported, including SHP099 and its derivatives,^{25,26} and IACS-15414.²⁷ The difference between the two classes of inhibitors is that the ASSIs bind to the open and activated SHP2, while the allosteric inhibitors bind to the closed and inactive SHP2. Despite these concerted efforts, no SHP2 targeting drugs have reached the clinic yet. Since activation of SHP2 involves its interaction with pTyr via the two SH2 domains, the abundant form of the SHP2 molecules in cancers with hyperactive Tyr kinase signaling is likely to be in an open and active conformation. As such, active-site inhibitors are likely to be more effective in cancers with hyperactive Tyr kinase signaling since they bind to the open and active SHP2. In line with this concept, we have recently reported an active site SHP2 inhibitor named CNBDA that showed promising antibreast cancer cell effects.²⁴ In the current study, we report modifications to CNBDA, which enhanced its antibreast cancer cell effects.

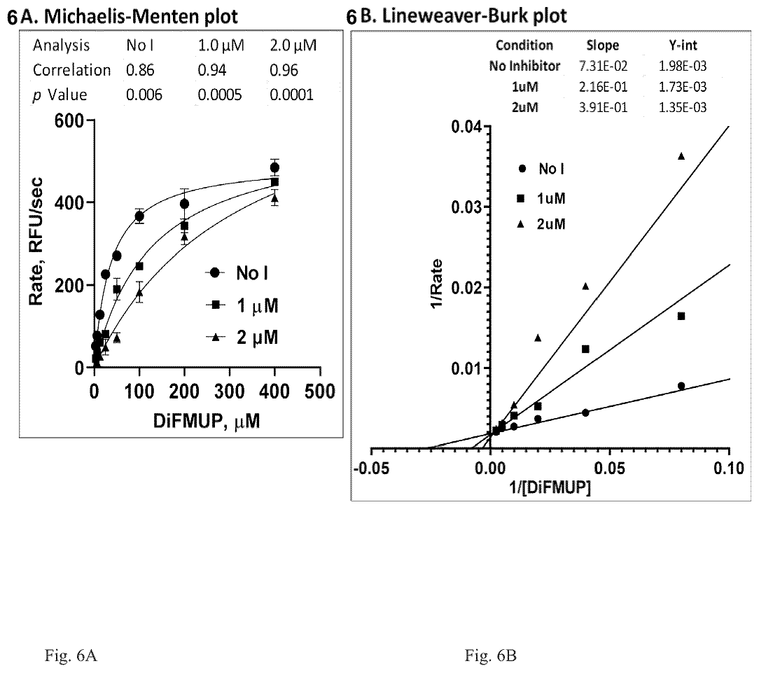

We have previously reported the design, synthesis, and characterization of a novel active site SHP2 inhibitor named CNBDA that has three polar carboxylic groups.²⁴ Although CNBDA showed effectiveness in inhibiting the SHP2 enzyme activity under in vitro conditions, the presence of three polar groups makes it less useful for future in vivo studies. We thus made two modifications that reduce the number of carboxylic groups from three to two. Furthermore, we have replaced one of the carboxylic groups with an acetic acid group in one of the modified structures. By doing so, we designed two derivatives, which are named CNBCA (3′-(3-carboxypropyl)-4′-(nonyloxy)-[1,1′-biphenyl]-4-carboxylic acid) and CNBBA (4-(4′-(carboxymethyl)-4-(nonyloxy)-[1,1′-biphenyl]-3-yl) butanoic acid). The aliphatic nonane group that was included in the parent compound to enhance cellular uptake was maintained in both CNBCA and CNBBA. The structure of the parent compound CNBDA is shown in FIG. 1 and the two compounds of this invention are shown in FIG. 6A.

We first used in silico molecular docking to predict similarities and differences in interaction properties between the parent compound CNBDA and the two newly designed compounds. We used the molecular modeling program Glide (Schrodinger) with induced-fit docking and binding energy calculation capabilities for these predictions.²⁸ Each molecule was docked into the SHP2 active site, using the crystal structure of the PTP domain (PDB: 4PVG) that was solved in complex with the active site inhibitor 11a-1.²¹ The docking results showed CNBCA making nine interactions with the SHP2 active site mediated by H-bonding, π-cation, and ionic interactions, while CNBBA making six interactions mediated by H-bonding, ionic interaction, and pi-pi stacking. The aliphatic nonane group is exposed to the solvent, suggesting minimal participation in the binding of CNBCA and CNBBA to the active site. We also docked each modified compound to the SHP1 active site (PDB: 1GWZ)²⁹, the close structural homologue of SHP2, for the purpose of comparison.

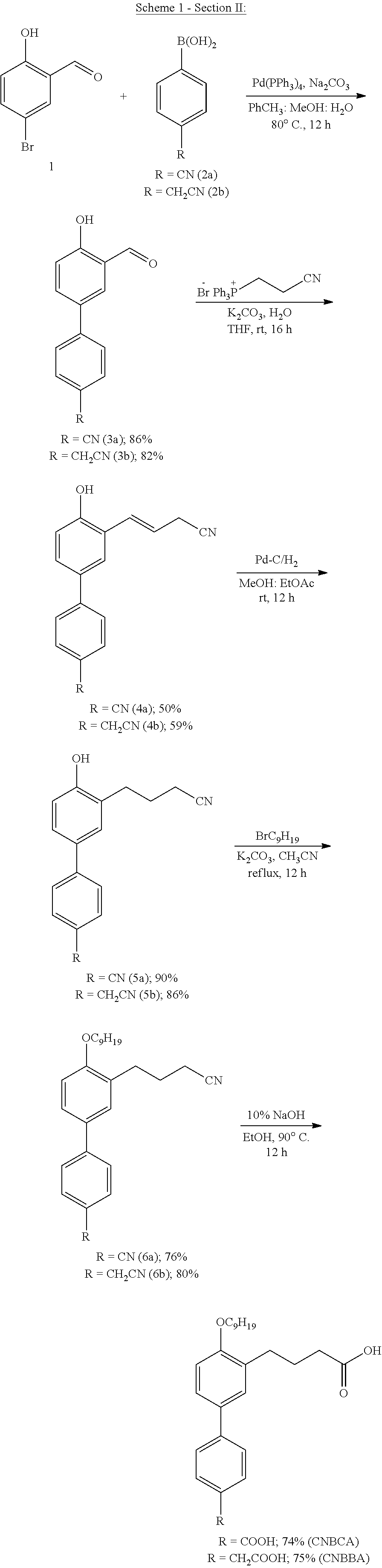

SHP1 is a 597 amino acid protein encoded by the PTPN6 gene (NCBI accession ID: XP_054228689.1). The docking data predicts very few interactions with the SHPI active site, suggesting that the two derivatives of this invention, namely CNBCA and CNBBA, make stronger interactions with the SHP2 active site but very weak interactions with the SHP1 active site. They also suggest that the interaction of CNBCA to the SHP2 active site is comparable to that of the parent compound CNBDA.
Synthesis Scheme. The synthesis scheme for CNBCA and CNBBA can be found in Scheme 1-Section II.

### CNBCA and CNBBA Inhibit the SHP2 Enzyme Activity.

After completion of synthesis, both CNBCA and CNBBA were purified on silica gel, and purity was determined by HPLC. The purified compounds were then tested for their effect on the SHP2 enzyme activity and on the activity of the selected control PTPs (SHP1 and PTP1B) by the phosphotyrosyl 30,31 which also made nine similar interactions.²⁴

As outlined in the synthesis scheme shown above (i.e. Scheme I Section II), the synthesis of CNBCA began with Suzuki cross-coupling reaction between 5-bromosalicylaldehyde (1) and 4-cyanophenylboronic acid (2) using Pd(PPh₃)₄ (5 mol %) and Na₂CO₃ in PhCH₃:MeOH:H₂O heated at 80° C. for 12 h to furnish 3a in 86% yield. Further, the Wittig olefination reaction was optimized using (2-cyanoethyl) triphenylphosphonium bromide, which was unstable with other bases. When three carbon-containing (2-cyanoethyl) triphenylphosphonium bromide salt was treated with K₂CO₃ in combination with one equivalent of water in THF, it generated stable Witting aldehyde. Addition of aldehyde 3a to that and stirring at room temperature for 16 h provided olefin 4a in 50% yield. Next, the double bond was reduced using Pd—C/H₂ to furnish saturated adduct 5a in 90% yield. This was O-alkylated using 1-bromononane to obtain 6a with 75% yield. At the end, both nitrile groups were hydrolyzed using NaOH in ethanol to furnish CNBCA in 74% yield.

For the synthesis of CNBBA, 5-bromosalicylaldehyde (1) was reacted with 4-(4,4,5,5-tetramethyl-1,3,2-dioxaborolan-2-yl) benzeneacetonitrile (2b) under Suzuki cross-coupling conditions to give 3b at 82% yield. Next, a similar synthetic sequence to that of CNBCA was followed (hydrogenation, O-alkylation, hydrolysis) to obtain CNBBA with 75% yield. The formation of CNBCA and CNBBA was confirmed by NMR and mass spectroscopy analysis.

After completion of synthesis, both CNBCA and CNBBA were purified on silica gel, and purity was determined by HPLC. The purified compounds were then tested for their effect on the SHP2 enzyme activity and on the activity of the selected control PTPs (SHP1 and PTP1B) by the phosphotyrosyl 30,31 which also made nine similar interactions.²⁴

### Phosphatase (PTPase) Assay.

First, the GST fusion of the PTP domains of SHP2, SHP1, and PTP1B were produced and purified as reported by us previously.³² The reactions were performed in a buffer containing 10 mM Tris-HCl (pH 7.2), 100 mM NaCl, 1 mM EDTA, 1 mM dithiothreitol (DTT), and 0.01% Tween-20. Briefly, CNBCA or CNBBA was added first in a serial dilution ranging from 100 μM to 97 nM followed by the enzymes (SHP2, SHP1, or PTP1B) to a final concentration of 1 nM. After 5 min of incubation at room temperature, the reactions were started by adding the artificial substrate DiFMUP (6,8-difluoro-4-methylumbelliferyl phosphate) to a final concentration of 20 μM for SHP2, 35 μM for SHP1, and 10 μM for PTP1B in a reaction volume of 100 μL; variations in DiFMUP concentrations reflect the reported Km values for the respective PTPs.³³ The complete mixture was incubated at 37° C. for 20 min, and fluorescence intensity was measured by the Synergy 4 plate reader at the excitation and emission wavelengths of 360 and 460 nm, respectively. The half inhibitory concentration (IC₅₀) value of each compound was calculated and plotted using Graphpad Prism software. The results showed inhibition of the SHP2 enzyme activity by CNBCA with an IC₅₀ of 0.87 μM and by CNBBA with an IC₅₀ of 5.1 μM (Table 1). While CNBCA showed significant improvement in potency over that of the parent compound (0.87 μM versus 5.0 μM), CNBBA exhibited similar potency as that of the parent compound (5.1 μM versus 5.0 μM). Comparative fold difference calculations showed that CNBCA is better than the parent compound CNBDA and the other new compound (CNBBA) by 5.7 fold and 5.8 fold, respectively.

Table 3 shows the inhibitory effect of CNBCA and CNBBA on the Enzyme Activity of SHP2.

TABLE 3

| | SHP2 IC₅₀, μM | fold difference |
|---|---|---|
| CNBCA | 0.87 | 1.0 |
| CNBBA | 5.1 | 5.8 |
| CNBDA | 5.0 | 5.7 |

We have previously shown that the parent compound CNBDA is selective for inhibition of SHP2 over that of SHPI by approximately 25 fold.²⁴ To test whether the relatively potent inhibitor CNBCA is also selective, we conducted comparative inhibition studies against the close structural homologue SHP1 and the ubiquitously expressed PTP1B, using the PTPase assay. The GST fusion PTP domains of SHP1 and PTP1B were expressed and purified in a fashion similar to that of SHP2. The results showed less effectiveness of CNBCA against SHP1 and PTP1B as evidenced by the significantly higher IC₅₀ values, 34.6 μM and 25.0 μM, respectively (Table 4). Calculation of selectivity ratios using

Table 4 shows the comparative IC₅₀ values of the most potent derivative CNBCA against SHP2 and two other PTPs, SHP1 and PTP1B, the Close Structural Homologue and the Most Ubiquitous PTP, respectively.

TABLE 4

| PTP | IC₅₀, μM | selectivity ratio |
|---|---|---|
| SHP2 | 0.87 | 1.0 |
| SHP1 | 34.6 | 39.77 |
| PTP1B | 25.0 | 28.73 |

The SHP2 IC₅₀ as a reference showed that CNBCA is more selective to SHP2 than for SHP1 and PTP1B by 39.77-fold and 28.73 fold, respectively. Taken together, data in Table 3 and Table 4 suggest that CNBCA is more potent and selective than parent compound CNBDA.

### CNBCA is a Competitive SHP2 Inhibitor

As suggested by the findings of molecular docking studies, CNBCA is designed to bind to the SHP2 active site. As such, its mechanism of inhibition is expected to be through competitive binding to the active site. To verify this point, we conducted enzyme kinetic studies using the artificial substrate DiFMUP in the presence and absence of CNBCA. Briefly, 400 μM DiFMUP in the PTPase buffer was added to the 12th well in the 96-well plate and then serially diluted to as low as 3.12 μM in the successive wells. After adding the enzyme to a final concentration of 1.0 nM in a total volume of 100 μL, the fluorescence of the enzyme reaction rate was measured for a total of 3 min at 10 s interval in the Synergy 4 plate reader at the excitation and emission wavelengths of 360 and 460 nm, respectively. The data was transformed into relative fluorescence unit (RFU) per second and plotted using the Graphpad software to produce the Michaelis-Menten and the Lineweaver-Burk plots. The Michaelis-Menten plot showed the dependence of the enzyme rate on substrate concentration, which was maximized as the DiFMUP concentration became saturating (FIG. 6A). Addition of CNBCA at 1.0 μM concentration shifted the rate to the right, and this effect was enhanced by increasing the amount to 2.0 μM, suggesting the occurrence of competitive inhibition. Calculation of the Pearson correlation coefficients (r) showed the significance of the findings. A Lineweaver-Burk or double-reciprocal plot of the same data showed a shift to the left (increased DiFMUP) to achieve maximum catalytic rate or Vₘₐₓ following the addition of CNBCA (FIG. 6B). Accordingly, the calculated Kₘ values were 36.05, 124.6, and 290.6 μM in the absence and in the presence of 1.0 and 2.0 μM CNBCA, respectively. Overall, the data set forth herein shows that CNBCA is a competitive inhibitor.

### CNBCA Binds to Full-Length SHP2 and Inhibits Enzyme Activity

Because the PTPase data described in Tables 3 and 4 were performed using isolated PTP domains lacking the regulatory SH2 domains, it was necessary to demonstrate whether CNBCA is also effective against full-length SHP2 (FL-SHP2). First, we used the cellular thermal shift assay (CETSA) to show whether CNBCA binds to endogenous full-length SHP2 under cellular contexts. The CETSA assay is based on the principle that specific inhibitors bind to a target protein and confer relative stability from heat-induced denaturation and precipitation when compared to a nontarget protein present in the same milieu.³⁴ Accordingly, we performed CETSA to test the effect of CNBCA on the SHP2 protein stability and compared it with the nontarget PTP1B protein as described in the materials and methods.

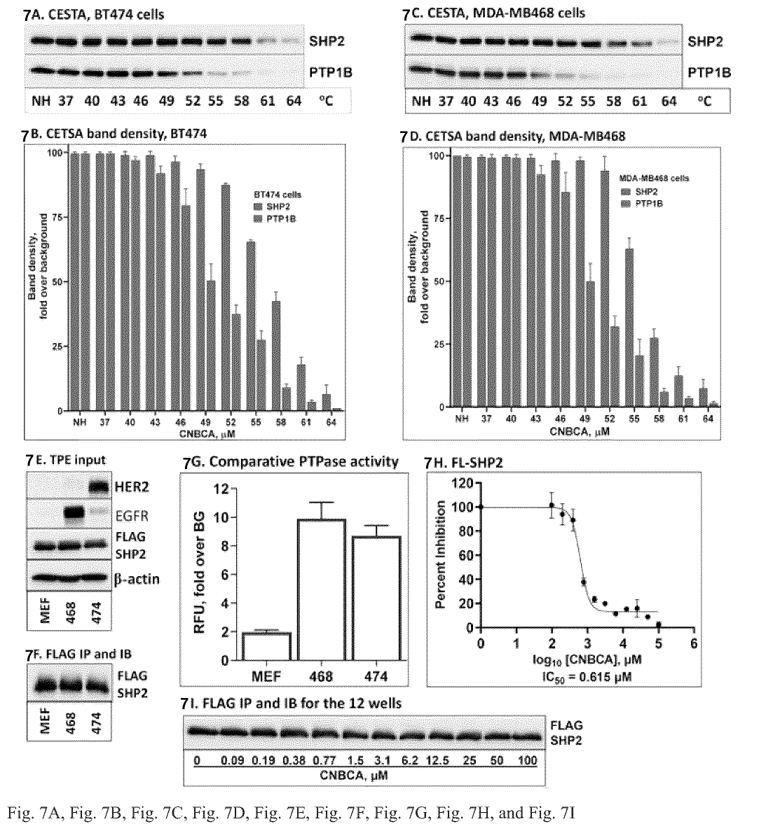

After heat treatment, supernatants were analyzed by immunoblotting (IB) for SHP2 and the nontarget phosphatase PTP1B. The results showed stabilization of SHP2 in solution by CNBCA treatment, but this effect was not as robust for PTP1B as evidenced by differences in signal intensity as the temperature of heat treatment increased (FIG. 7A and FIG. 7C) To provide semiquantitative data, band densities of both SHP2 and PTP1B were measured and presented as bar graph. The results showed stabilization of SHP2 in solution to nearly 100% at 49° C., to 95% at 52° C., to 70% at 55° C., and to 45% at 58° C. On the other hand, the nontarget protein PTP1B was reduced to approximately 50% at 49° C., to 25% at 55° C., and to less than 10% at 58° C. (FIG. 7B and FIG. 7D). These findings clearly show that CNBCA differentially stabilizes SHP2 in solution by binding to the full-length protein (FL-SHP2). On the other hand, CNBCA is less effective in stabilizing the nontarget protein PTP1B, suggesting its specificity to SHP2.

To show whether CNBCA also inhibits cellular SHP2, we first compared the enzyme activity of FL-SHP2 expressed in fibroblasts and BC cells as FLAG-tagged protein.³⁵ To confirm efficiency of the expression, we conducted IB analysis of total protein extracts (TPE) prepared from the noncancerous MEF cells (mouse embryo fibroblasts), the MDA-MB468 BC cells that overexpress EGFR, and the BT474 BC cells that overexpress HER2. The results showed the efficient expression of FL-SHP2 as determined by anti-FLAG IB (FIG. 7E). We also performed HER2 and EGFR IB analyses, which confirmed overexpression of these RTKs in the BC cells, but not in the MEF cells. IB for β-actin was used as a loading control, which appears to be comparable in all lanes. Next, FL-SHP2 was immunoprecipitated (IP) with anti-FLAG antibody from the TPEs of the three cells (FIG. 7F) and used for determining the activation state while still bound to protein-A sepharose beads, as described in the materials and methods. As shown in FIG. 7G, SHP2 expressed in BC cells was enzymatically more active by at least 5 fold than SHP2 expressed in the noncancerous MEF cells, suggesting that hyperactive Tyr kinase signaling activates the PTPase function of SHP2. We thus used SHP2 expressed in Breast Cancer (BC) cells to test the inhibitory effect of CNBCA on FL-SHP2. Accordingly, FL-SHP2 was immunoprecipitated from the TPE of the MDA-MB468 cells with anti-FLAG antibody, divided equally into 12 wells, treated with CNBCA in a serial dilution, ranging from 95 nM to 100 μM, incubated at room temperature for 5 min, and then tested by a PTPase assay as described above for the PTP domain. The results showed inhibition of FL-SHP2 by CNBCA with an IC₅₀ of 0.615 μM (FIG. 7H), which is slightly better than the IC₅₀ observed with the PTP domain (see Table 3). IB analysis of immunoprecipitates from each well showed comparable protein levels in all lanes (FIG. 7I). However, it was not possible to estimate the concentration of FL-SHP2 in each well due to the nature of the experiment. Nonetheless, these findings suggest that CNBCA is also effective against FL-SHP2, complementing the CETSA results.

### CNBCA Downregulates Basal Signaling in Breast Cancer Cells

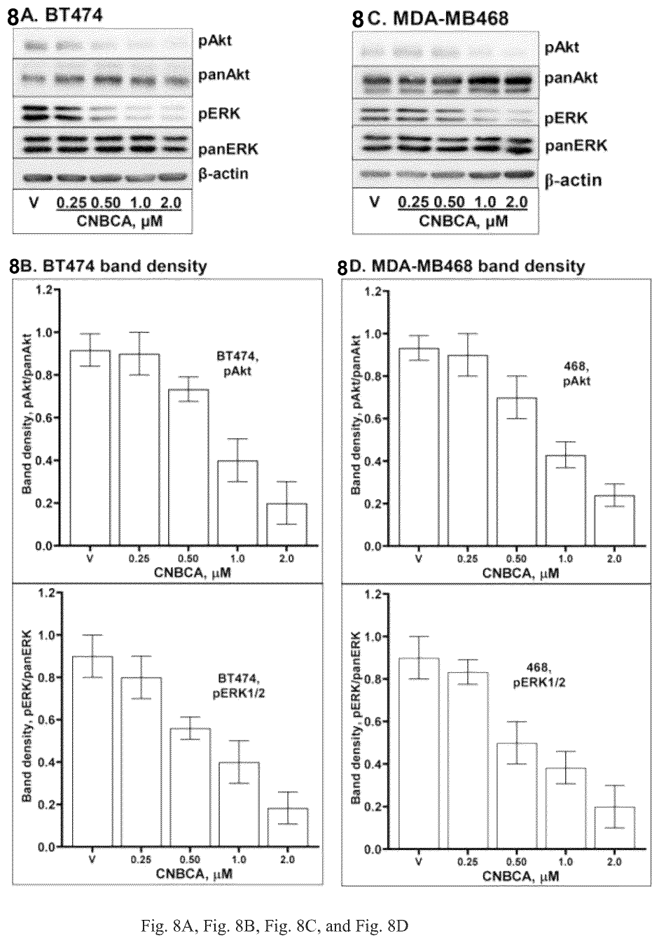

Since SHP2 is an essential mediator of mitogenic and cell survival signaling in breast cancer cells, we determined the effect of CNBCA treatment on activation of ERK 1/2 and Akt in the HER-2 positive BT474 and in the triple negative MDA-MB468 cell lines. Cells growing in 2D culture were treated with varying concentrations of CNBCA, ranging from 0.250 to 2.0 μM, for 48 h (hour), and total cell lysates were analyzed by immunoblotting (IB) with antibodies that recognize the activated forms of ERK1/2 (PERK 1/2) and Akt (pAkt). The results showed downregulation of basal pERK1/2 and pAkt levels by CNBCA treatment in a concentration dependent manner (FIG. 8A and FIG. 8C) IB analysis for total Akt (panAkt), total ERK1/2 (panERK), and β-actin showed similar total protein levels in all lanes. Band density measurements from three independent experiments showed that CNBCA inhibited SHP2 mediated Akt and ERK1/2 activation in the BT474 and MDA-MB468 cells with an approximate IC₅₀ of 1.0 μM (FIG. 8B and FIG. 8D). These findings suggest that CNBCA effectively downregulates SHP2 mediated signaling in BC cells.

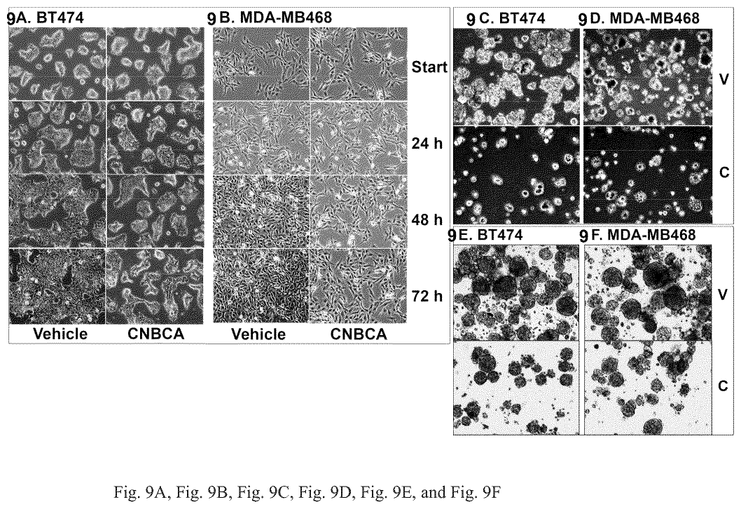

Since SHP2 is essential for the growth and transformation of BC cells,^{8,9,11} we determined the effect of SHP2 inhibition with CNBCA in 2D and 3D culture systems. For the effect on cell growth, BC cells (BT474 and MDA-MB468) were seeded in 2D culture and treated with a single concentration of CNBCA (500 nM), the concentration that provided a 50% inhibition of SHP2 mediated signaling in BT474 cells. Images were collected at the start and every 24 h for a total of 72 h (3 days). The results showed growth of the vehicle treated cells to confluency during this period, but the CNBCA treated cells were still half-confluent, suggesting suppression of cell proliferation (FIG. 9A and FIG. 9B). For effect on anchorage independent growth, approximately 10⁶ cells were seeded in soft agar as described previously,²⁴ and treated with CNBCA at 500 nM concentration for 10 days with refeeding and adjustment of compound concentration. Images collected at the 4× microscopic objective showed that the vehicle treated cells formed more and larger colonies, while the CNBCA treated cells formed fewer and smaller colonies (FIG. 9C and FIG. 9D). Finally, the effect of CNBCA on cancer stem cell properties was determined by the suspension assay, in which only cells with stem-like properties only can grow and form mammospheres.^{7,24,36} Approximately 10⁶ cells were seeded in nonadherent 6 cm plates and treated with CNBCA (500 nM) for 10 days with complementation of media and drug concentration. The results showed formation of larger mammospheres by the vehicle treated cells and fewer and smaller mammospheres by the CNBCA treated cells (FIG. 9E and FIG. 9F). These finding suggest that CNBCA is effective in suppressing the cancer stem cell properties of BC cells.

It will be understood by those persons of ordinary skill in the art that this invention provides modification of the parent active site SHP2 inhibitor CNBDA to produce derivatives with improved potency. Of the two derivatives, CNBCA showed significantly improved potency in inhibiting the PTPase activity of SHP2. More specifically, CNBCA inhibited the SHP2 PTPase activity with an IC₅₀ of 0.87 μM, which is better than the parent compound that inhibited the SHP2 PTPase activity with an IC₅₀ of 5.0 μM. In addition, the PTPase assay data showed that CNBCA is more selective to SHP2 than the control PTPs (SHP1 and PTP1B), and is a competitive inhibitor. Furthermore, CNBCA binds to FL-SHP2 and inhibits its enzyme activity, downregulates SHP2 mediated signaling, and suppresses the growth and transformation phenotypes of breast cancer cells. Overall, data reported here show that CNBCA that lacks one of the butyric acid groups of the parent compound functions with better potency in both inhibiting SHP2 enzyme activity and suppressing breast cancer cell phenotypes.

### Experimental Section II

### Molecular Docking

To predict the binding properties of the newly designed compounds, we employed in silico molecular docking to predict similarities and differences in interaction properties between the parent compound CNBDA and the two newly designed compounds, using the molecular modeling program Glide (Schrodinger) with induced-fit docking and binding energy calculation capabilities.²⁸ Each molecule was docked into the SHP2 active site, using the crystal structure of the PTP domain (PDB: 4PVG) that was solved in complex with the active site inhibitor 11a-1.²¹ The compounds were also docked into the SHP1 active site using the previously published PTP structure (PDB: 1GWZ) for comparison. Since the compounds are designed to bind to the active site of SHP2, the 20×20×20 Å³ grid box was placed around the point defined by the sulfur atom of the catalytic cysteine (C459 for SHP2 and C455 for SHP1). Prime MM-GBSA calculations were used to predict changes in free energy of binding denoted as ΔG. The two-dimensional interaction map was generated from the PDB file of the docked poses, using the MAESTRO 2-D sketcher of Glide.

### Chemistry

For organic synthesis, reagents and solvents were purchased from Sigma-Aldrich (Sigma-Aldrich, TCI, Oakwood, Enamine, and ChemImpex) and were used without additional purifications. ¹H and ¹³C NMR spectra were recorded on Jeol 400 spectrometers with TMS or residual solvent as standard Column chromatography was performed on silica gel (100-200 mesh) using a proper eluent. Thin-layer chromatography (TLC) analysis was performed on precoated silica gel 60 F254 plates. Visualization on TLC was achieved by the use of UV light (254 nm). NMR spectra were recorded in chloroform-d, methanol-d₄, and DMSO-d₆ at 400 MHz for ¹H NMR spectra and 100 MHz for ¹³C NMR spectra. Chemical shifts were quoted in parts per million (ppm) referenced to the appropriate solvent peak or 0.0 ppm for tetramethylsilane. The following abbreviations were used to describe peak splitting patterns when appropriate: br=broad, s=singlet, d=doublet, t=triplet, q=quartet, p=pentet (quintet), dd=doublet of doublet, td=triplet of doublet, m=multiplet. Coupling constants (J) are reported in hertz (Hz). ¹³C NMR chemical shifts were reported in ppm referenced to the center of a triplet at 77.0 ppm of chloroform-d, 49.0 ppm for methanol-d₄ and 40.0 ppm center for DMSO-d₆. HRMS spectra were recorded using Quadrupole and Orbitrap LC-MS/MS techniques. The purity of the final products was determined by HPLC, which was performed on an Agilent 1100 Series flexible pump, a vial sampler, and a multicolumn thermostat fitted with a New Phenomenex Hyperclone MOS C8 column 5 μm, 4.6 mm×150 mm HPLC 00F-4359-E0, a diode array detector, and a 6125B MSD single quadrupole detector (eluent A, 0.1% formic acid in water; eluent B, acetonitrile). The method composition for the HPLC was: 2 min (A: 98.00%, b: 2.00%), 25 min (A: 40.00%, B: 60.00), 27 min (A: 5.0%, B: 95.0%), 29 min (A: 5.0%, B: 95.0%), 30 min (A: 98.0%, B: 2.0%). These analyses showed that the final compounds were >95% pure as determined by reverse phase HPLC (λ 250 nm).

### 3′-Formyl-4′-hydroxy-[1,1′-biphenyl]-4-carbonitrile (3a)

Under nitrogen atmosphere, a mixture of 5-Bromosalicylaldehyde (1, 804 mg, 4.0 mmol), 4-cyanophenylboronic acid (2a, 705, 4.8 mmol), Pd(PPh₃)₄ (231 mg, 5 mol %), Na₂CO₃ (848 mg, 8.0 mmol) in toluene (10 mL), water (5 mL), and methanol (5 mL) was refluxed for 12 h at 80° C. After completion of the reaction (by TLC), the mixture was cooled to room temperature, the solvent was evaporated, and the crude residue was quenched with saturated ammonium chloride and extracted with ethyl acetate (50 mL×3). The extract was washed with water and brine, dried over NaSO, and evaporated. The residue was purified by column chromatography over silica gel (n-hexane/ethyl acetate=6:4) to give 3a as a white solid (776 mg, 86%). ¹H NMR (400 MHz, CDCl₃) δ: 11.10 (s, 1H), 10.00 (s, 1H), 7.76 (m, J=13.3, 7.5 Hz, 3H), 7.68 (t, J=9.6 Hz, 3H), 7.13 (d, J=8.3 Hz, 1H). ¹³C NMR (100 MHz, CDCl₃) δ: 196.4, 162.0, 143.7, 143.5, 135.5, 132.9, 132.8, 132.2, 127.9, 127.1, 120.8, 118.7.

(E)-3′-(3-Cyanoprop-1-en-1-yl)-4′-hydroxy-[1,1′-biphenyl]-4-carbonitrile (4a). The Wittig olefination reaction was optimized and used for the synthesis of 4a. Under nitrogen atmosphere to around-bottom flask charged with (2-cyanoethyl) triphenylphosphonium bromide (1.3 g, 3.32 mmol, 1.5 equiv), K₂CO₃ (453 mg, 3.32 mmol, 1.5 equiv), and H₂O (45 μL, 2.19 mmol, 1 equiv), the resulting mixture stirred at room temperature for 20 min. To that aldehyde 3a (490 mg, 2.19 mmol, 1 equiv) was added, and the resulting mixture stirred at room temperature for 16 h. After completion of reaction (by TLC), it was quenched with water and extracted with ethyl acetate (50 mL×3), organic layer was dried over sodium sulfate and evaporated on rotavapor. The crude residue was purified by column chromatography over silica gel (n-hexane/ethyl acetate=1:1) to give 4a as a white solid (280 mg, 50%). ¹H NMR (400 MHz, DMSO-d₆) δ 10.19 (s, 1H), 7.94-7.72 (m, 5H), 7.52 (dd, J=8.4, 1.6 Hz, 1H), 6.98 (d, J=8.5 Hz, 1H), 6.93 (d, J=16.0 Hz, 1H), 6.42 (dt, J=16.0 Hz, 1H), 3.56 (d, J=5.5 Hz, 2H). ¹³C NMR (100 MHz, DMSO-d₆) δ: 156.0, 144.9, 133.1, 129.6, 128.5, 128.0, 127.3, 126.2, 123.8, 119.8, 119.6, 119.2. 116.9, 109.4, 20.8.

3′-(3-Cyanopropyl)-4′-hydroxy-[1,1′-biphenyl]-4-carbonitrile (5a). To a stirred solution of 4a in ethyl acetate:methanol (1:1, 10 mL) was added Pd—C (5 mol %), and the mixture was stirred under hydrogen atmosphere at room temperature for 12 h. After completion of the reaction by TLC, the mixture was filtered off and solvent was evaporated on rotavapor. The crude residue was purified by column chromatography over silica gel (n-hexane/ethyl acetate=3:1) to give 5a as a sticky oil (227 mg, 90%). ¹H NMR (400 MHz, CDCl₃) δ 7.68 (dd, J=8.2, 2.3 Hz, 2H), 7.64-7.57 (2 H), 7.38-7.32 (m, 2H), 6.84 (dd, J=8.1, 2.3 Hz, 2H), 5.23 (s, J=1.8 Hz, 1H), 2.90-2.76 (t, 2H), 2.38 (t, J=7.1, 2.4 Hz, 2H), 2.04 (m, J=9.0, 4.5 Hz, 2H). ¹³C NMR (100 MHz, CDCl₃) δ: 154.4, 145.0, 132.6, 132.0, 129.5, 127.1, 126.7, 119.6. 119.0, 116.0, 110.2, 29.3, 25.3, 16.7.

3′-(3-Cyanopropyl)-4′-(nonyloxy)-[1,1′-biphenyl]-4-carbonitrile (6a). Under nitrogen atmosphere, a mixture of 5a (100 mg, 0.381 mmol) and K₂CO₃ (106 mg, 0.762 mmol) in anhydrous acetonitrile 10 mL was stirred at rt for 10 min, and 1-Bromononane (99 mg, 0.476 mmol) was added and the resulting mixture was refluxed for 12 h. After completion by TLC, mixture was cooled down to room temperature and then diluted with water and extracted with ethyl acetate (50 mL×3). The extract was washed with water and brine, dried over Na₂SO₄, and evaporated. The crude oil was purified by column chromatography over silica gel (n-hexane/ethyl acetate, 3:1) to give 6a as a white solid (115 mg, 76%). ¹H NMR (400 MHz, CDCl₃) δ 7.69 (d, J=8.3 Hz, 2H), 7.64 (d, J=8.2 Hz, 2H), 7.44 (dd, J=8.4, 2.0 Hz, 1H), 7.38 (d, J=1.8 Hz, 1H), 6.93 (d, J=8.5 Hz, 1H), 4.02 (t, J=6.4 Hz, 2H), 2.85 (t, J=7.3 Hz, 2H), 2.35 (t, J=7.1 Hz, 2H), 2.01 (q, J=7.2 Hz, 2H), 1.89-1.77 (q, 2H), 1.53-1.43 (m, 2H), 1.36-1.24 (m, 10H), 0.89 (t, J=6.5 Hz, 3H). ¹³C NMR (100 MHz, CDCl₃) δ: 157.6, 145.12, 132.6, 131.0, 129.0, 127.0, 126.7, 119.7, 119.0, 68.1, 31.9, 29.7, 29.5, 29.3, 29.3, 29.2, 26 1, 25.4, 22.7, 16.7, 14.1.

3′-(3-Carboxypropyl)-4′-(nonyloxy)-[1,1′-biphenyl]-4-carboxylic Acid (CNBCA). To a 1 N NaOH solution (3 mL) was added 6a (50 mg, 0.128 mmol) in EtOH (3 mL). The resulting mixture was refluxed for 12 h, and when TLC (60% ethyl acetate/hexane) showed that the reaction was completed; the mixture was concentrated under reduced pressure to remove ethanol. The resulting aqueous solution was cooled in an ice bath and made acidic (pH 1) with 1 N HCl to give a precipitate. The aqueous suspension was extracted with ethyl acetate (30 mL×3). The organic phase was washed with saturated brine (50 mL), dried over sodium sulfate, and filtered, and the filtrate was concentrated under reduced pressure to give a white solid. It was purified by column chromatography over silica gel (n-hexane/ethyl acetate, 1:1) to give CNBCA as a white solid (41 mg, 74%). ¹H NMR (400 MHz, methanol-d4) δ 8.05 (d, J=8.0 Hz, 2H), 7.69 (d, J=7.9 Hz, 2H), 7.51 (d, J=8.5 Hz, 1H), 7.47 (s, 1H), 7.01 (d, J=8.5 Hz, 1H), 4.04 (t, J=6.2 Hz, 2H), 2.74 (t, J=7.4 Hz, 2H), 2.32 (t, J=7.4 Hz, 2H), 1.94 (p, J=7.5 Hz, 2H), 1.88-1.79 (m, 2H), 1.53 (dd, J=14.6, 6.9 Hz, 2H), 1.43-1.26 (m, 10H), 0.91 (t, J=6.2 Hz, 3H). ¹³C NMR (100 MHz, methanol-d₄) δ: 177.5, 169.9, 158.8, 147.0, 133.0. 131.7, 131.3, 129.9, 129.8, 127.4, 127.2, 112.7, 69.0, 34.5, 33.0, 30.9, 30.7, 30.5, 30.5, 30.4, 27.4, 26.4, 23.8, 14.5. HRMS (ESI) [M−H]⁻: m/z caldt for C₂₆H₃₄O₅: 424.5564; found: 424.5564.

2-(3′-Formyl-4′-hydroxy-[1,1′-biphenyl]-4-yl) Acetonitrile (3b). According to the procedure described for the preparation of 3a, the same procedure was followed for 3b; under a nitrogen atmosphere, a mixture of 5-bromosalicylaldehyde (1, 1 g, 0.497 mmol), 4-(4,4,5,5-tetramethyl-1,3,2-dioxaborolan-2-yl) benzeneacetonitrile (2b, 1.45 g, 0.597 mmol), Pd(PPh₃)₄ (287 mg, 5 mol %), Na₂CO₃ (1.0 g, 0.994 mmol) in toluene (10 mL), water (5 mL), and methanol (5 mL) was refluxed for 12 h at 80° C. to give 3b as a yellow solid (950 mg, 82%). ¹H NMR (400 MHz, CDCl₃) δ 11.02 (s, J=2.7 Hz, 1H), 9.97 (s, J=2.5 Hz, 1H), 7.75 (dd, J=5.6, 2.4 Hz, 2H), 7.60-7.52 (m, 2H), 7.41 (d, J=6.1 Hz, 2H), 7.08 (dd, J=9.1, 2.6 Hz, 1H), 3.80 (d, J=2.0 Hz, 3H). ¹³C NMR (100 MHz, CDCl₃) δ: 196.5, 161.2, 139.2, 135.6, 132.2, 131.8, 129.0, 128.6, 127.2, 120.7, 118.3, 117.7, 23.3.

(E)-4-(4′-(Cyanomethyl)-4-hydroxy-[1,1′-biphenyl]-3-yl) But-3-enenitrile (4b). According to the procedure described for the preparation of 4a, the same procedure was followed for 4b; under nitrogen atmosphere to a round-bottom flask charged with (2-cyanoethyl) triphenylphosphonium bromide (521 mg, 1.31 mmol, 1.3 equiv), K₂CO₃ (210 mg, 1.51 mmol, 1.3 equiv), and H₂O (19 μL, 1.0 mmol, 1 equiv), resulting mixture stirred at room temperature for 20 min. To that aldehyde 3b (240 mg, 1.0 mmol, 1 equiv) was added, and the resulting mixture was stirred at room temperature for 16 h to obtain 4b as a light yellow solid (165 mg, 59%). ¹H NMR (400 MHz, CDCl₃) δ: 7.52 (d, J=6.9 Hz, 3H), 7.38-7.30 (m, 3H), 6.98 (d, J=16.1 Hz, 1H), 6.87 (d, J=8.3 Hz, 1H), 6.21 (dt, J=15.7, 5.7 Hz, 1H), 6.07 (s, J=12.2 Hz, 1H), 3.78 (s, 2H), 3.32 (d, J=5.7 Hz, 2H). ¹³C NMR (100 MHz, CDCl₃) δ: 153.2, 140.3, 132.8, 129.6, 128.3, 128.2, 127.7, 127.3, 126.4, 123.3, 122.7, 118.4, 118.0, 117.6, 116.5, 23.2, 21.2.

4-(4′-(Cyanomethyl)-4-hydroxy-[1,1′-biphenyl]-3-yl) Butanenitrile (5b). To a stirred solution of 4b (90 mg, 0.328 mmol) in ethyl acetate:methanol (1:1, 12 mL) was added Pd—C (5 mol %), and the mixture was stirred under hydrogen atmosphere at room temperature for 12 h. After completion of the reaction by TLC, the mixture was filtered off and solvent was evaporated on rotavapor. The crude residue was purified by column chromatography over silica gel (n-hexane/ethyl acetate=3:1) to give 5b as a light yellow solid (78 mg, 86%). ¹H NMR (400 MHz, CDCl₃) δ 7.54 (d, J=8.1 Hz, 2H), 7.40-7.29 (m, 4H), 6.83 (d, J=8.1 Hz, 1H), 5.33 (s, J=5.9 Hz, 1H), 3.79 (s, 2H), 2.85 (t, J=7.4 Hz, 3H), 2.39 (t, J=7.1 Hz, 2H), 2.05 (p, J=7.3 Hz, 2H). ¹³C NMR (100 MHz, CDCl₃) δ: 153.7, 140.5, 132.9, 129.2, 128.3, 128.1, 127.3, 126.7, 126.4, 119.8, 117.9, 115 8, 29.3, 25.3, 23.3, 16.6.

4-(4′-(Cyanomethyl)-4-(nonyloxy)-[1,1′-biphenyl]-3-yl) Butanenitrile (6b). According to the procedure described for the preparation of 6a, the same procedure was followed for 6b; under nitrogen atmosphere, a mixture of 5b (35 mg, 0.126 mmol) and K₂CO₃ (22 mg, 0.158 mmol) in anhydrous acetonitrile 5 mL was stirred at room temperature for 10 min, to that 1-bromononane (33 mg, 0.158 mmol) was added, and the resulting mixture was refluxed for 12 h to furnish 6b as a yellow liquid (42 mg, 80%). ¹H NMR (400 MHz, CDCl₃) δ 7.55 (d, J=7.7 Hz, 2H), 7.39 (m, J=16.8, 8.8 Hz, 4H), 6.91 (d, J=8.4 Hz, 1H), 4.01 (t, J=6.4 Hz, 2H), 3 78 (s, 2H), 2.84 (t, J=7.2 Hz, 2H), 2.35 (t, J=7.1 Hz, 2H), 2.08-1.94 (q, 2H), 1.89-1.77 (q, 2H) 1.47 (m, J=14.1, 6.9 Hz, 2H), 1.29 (m, 10H), 0.89 (t, J=6.0 Hz, 3H). ¹³C NMR (100 MHz, CDCl₃) δ: 156.8, 140.6, 132.3, 128.9, 128.7, 128.3, 128.1, 127.3, 126.3, 119.8, 117.9, 111.5, 68.0, 31.9, 29.7, 29.5, 29.3, 29.3, 26.2, 25.5, 23.3, 22.7, 16.7.

4-(4′-(Carboxymethyl)-4-(nonyloxy)-[1,1′-biphenyl]-3-yl) Butanoic Acid (CNBBA). To a 1 N NaOH solution (3 mL) was added 6b (27 mg, 0.067 mmol) in EtOH (pH 3.0) (3 mL). The resulting mixture was refluxed for 12 h, and when TLC (60% ethyl acetate/hexane) showed that the reaction was completed, the mixture was concentrated under reduced pressure to remove ethanol. The resulting aqueous solution was cooled in an ice bath and made acidic (pH 1) with 1 N HCl to give a precipitate The aqueous suspension was extracted with an ethyl acetate (25 mL×3). The organic phase was washed with saturated brine (50 mL), dried over sodium sulfate, and filtered, and the filtrate was concentrated under reduced pressure to give a white solid. It was purified by column chromatography over silica gel (n-hexane/ethyl acetate, 1:1) to give CNBBA as a light yellow solid (24 mg, 75%). ¹H NMR (400 MHz, CDCl₃) δ 7.49 (d, J=7.6 Hz, 2H), 7.34 (m, J=8.8 Hz, 4H), 6.87 (d, J=8.2 Hz, 1H), 3.98 (t, J=6.3 Hz, 2H), 3.68 (s, 2H), 2.73 (t, J=7.3 Hz, 2H), 2.39 (t, J=7.1 Hz, 2H), 2.08-1.91 (q, 2H), 1.88-1.74 (q, 2H), 1.53-1.42 (m, 2H), 1.30 (m, J=20.7 Hz, 10H), 0.89 (t, J=6.1 Hz, 3H). ¹³C NMR (100 MHz, CDCl₃) δ: 179.9, 177.7, 156.6, 140.3, 132.9, 131.6, 129.9, 129.7. 129.0, 127.0, 125.7, 111.3, 68.0, 40.8, 33.3, 31.9, 29.6, 29.4, 29.3, 26.2, 24.6, 22.7, 14.0. HRMS (ESI) [M−H]⁻: m/z caldt for C₂₇H₃₆O₅: 438.5864; found: 438.5864.

### PTPase Assay:

We used the PTPase assay described previously^{30,31} to evaluate the effect of the newly synthesized compounds on the enzymatic activity of SHP2, SHP1, and PTP1B. The PTP domains of the indicated proteins were prepared as reported by us recently.³² The artificial PTP substrate DiFMUP (6,8-Difluoro-4-Methylumbelliferyl Phosphate) was purchased from Invitrogen. The purified proteins were dialyzed into a phosphatase buffer containing 10 mM Tris-HCl (pH 7.72), 100 mM NaCl, 1.0 mM EDTA, 1.0 mM dithiothreitol (DTT), and 0.01% Tween-20. Briefly, the PTPase reactions were performed in 100 μL volume containing 1 nM enzyme, inhibitors in serial dilutions (12 nM to 100 μM), and DiFMUP to a final concentration of 20 μM for SHP2, 35 μM for SHP1, and 10 μM for PTP1B. Differences in DiFMUP concentrations reflect the reported Km values for each PTP.³⁷ The mixtures were incubated at 37° C. for 20 min and transferred to 96 well plates, and the fluorescence intensity was measured by a Synergy 4 plate reader at the excitation and emission wavelengths of 360 and 460 nm, respectively. Graphpad Prism software was used to calculate the IC₅₀ values.

For determining the inhibitory effect of CNBCA on FL-SHP2, a FLAG-tagged protein ectopically expressed in the EGFR over-expressing MDA-MB468 cells^{8,15} was used as an enzyme. First, the protein was purified by immunoprecipitation with anti-FLAG antibody from total protein extracts (TPEs). Next, SHP2 bound to the beads was equally divided into 12 wells, mixed with varying concentration CNBCA prepared by serial dilution (95 nM-100 μM) in the PTPase buffer described above, and incubated at room temperature for 5 min to allow binding. The PTPase reaction was started by adding DiFMUP to a final concentration of 20 μM in approximately 120 μL of total volume, which includes the beads. After 20 min of incubation at 37° C., 100 μL of the supernatant from each sample was transferred to 96-well plates, and fluorescence intensity measure as above, using the Synergy 4 plate reader. The resulting data was then analyzed with the Graphpad Prism software to draw the graph and determine the IC₅₀ value.

To confirm that CNBCA competitively inhibits SHP2, we conducted enzyme kinetic studies using the SHP2 enzyme at 1.0 nM, DiFMUP in serial dilutions in 96-well plates, ranging from 1 to 1024 μM, and in the absence and presence of two different concentrations of CNBCA (1.0 and 2.0 μM). In these reactions, the enzyme was added last, and the fluorescence was read for 3 min at 10 s interval. Changes in fluorescence intensity were used to calculate the Kₘ of the SHP2 enzyme for DiFMUP in the presence and absence of CNBCA. The Graphpad Prism was used to prepare the Michaelis-Menten and Lineweaver-Burk plots and to determine the Km values.

### Cellular Thermal Shift Assay (CETSA)

For the CETSA study, total protein extracts (TPE) prepared from two breast cancer cells, the triple-negative MDA-MB468 and the HER2-positive JIMT-1 cells, were used. Briefly, TPE from the two cell lines were cleared by centrifugation, mixed with CNBCA at 100 μM concentration (the maximum amount used in the IC₅₀ studies), and incubated for 10 min at room temperature to allow binding. The mixture was then divided into 100 μL aliquots, treated with heat for 10 min at different temperatures, ranging from 37 to 64° C. that differ by 3° C. intervals, cooled on ice for 10 min, centrifuged at 20,000 g for 20 min to remove precipitated proteins, and the supernatants analyzed by immunoblotting (IB) for SHP2 and the nontarget PTP, PTP1B. Aliquots that were mixed with CNBCA, but not treated with heat were used as positive controls for comparison.

### Cells and Reagents:

Both the HER2-positive BT474 and the triple-negative MDA-MB468 breast cancer cell lines were purchased from the American Tissue Culture Collection (ATCC). While the BT474 cells were grown in RPMI, the MDA-MB468 cells were grown in Dulbecco's modified Eagle's medium (DMEM), both supplemented with 10% fetal bovine serum. The artificial substrate DiFMUP was purchased from Invitrogen, while glutathione-sepharose beads used for purification of GST fusion PTP domains were purchased from GE Healthcare. The antibodies used in this study, the antiphospho-ERK1/2, anti-phospho-Akt, and anti-panAkt antibodies, were from Cell Signaling biotechnology, the anti panERK2 antibody was from BD Biosciences, and the anti-β-actin antibody was from Sigma-Aldrich.

### Colony Formation Assay:

For colony formation studies, we used the soft agar assay, which keeps cells anchorage independently, allowing determination on the transformation phenotype of cancer cells, including breast cancer cells.^{11,35} For this assay, we first prepared a 5% stock suspension in PBS by boiling it in a microwave. Just before use, the solidified agar suspension was boiled and allowed to cool to approximately 45° C. in a water bath and added to cell suspensions in 2 mL of growth medium at a final concentration of 0.3%. The mixture was immediately poured into 6 cm culture plates, allowed to solidify for about 5 min at room temperature, and incubated in a cell culture incubator with 50% CO₂ at 37° C. for approximately 10 days. Vehicle or CNBDA at 500 nM was added to the cells before mixing with the agar, which was maintained for the 10 day incubation time. Colony formation was visualized by imaging under an Olympus IX71 microscope equipped with a DP80 camera.

### Mammosphere Formation Assay:

To further evaluate the anti-breast cancer cell effect of CNBCA, we treated cells seeded in a suspension culture known as the mammosphere assay. In this assay, cells with cancer stem cell properties can grow and form sphere-like cellular aggregates known as ammospheres.^{7,38} For this assay, approximately 10⁶ cells were seeded in serum-free DMEM containing 1 μg/mL hydrocortisone, 10 μg/mL insulin, 10 ng/mL EGF, 10 ng/mL FGF, 5 ng/ml heparin, and B27 (Invitrogen) in 6 cm ultralow adherence culture plates and then treated with vehicle or CNBCA at 500 nM final concentration for approximately 10 days. After these days, mammospheres were collected in smaller volume of media using centrifugation, and imaged using the 10× objective in an Olympus IX71 microscope equipped with DP30 camera. Representative images for each experimental group are used to show effectiveness of the anti-SHP2 compound CNBCA of this invention.

### REFERENCES SECTION II

(1) Agazie, Y.; Ischenko, I.; Hayman, M. Concomitant activation of the PI3K-Akt and the Ras-ERK signaling pathways is essential for transformation by the V-SEA tyrosine kinase oncogene. Oncogene 2002, 21 (5), 697-707.
(2) Agazie, Y. M.; Hayman, M. J. Molecular mechanism for a role of SHP2 in epidermal growth factor receptor signaling. Molecular and cellular biology 2003, 23 (21), 7875-86.
(3) Agazie, Y. M.; Movilla, N.; Ischenko, I.; Hayman, M. J. The phosphotyrosine phosphatase SHP2 is a critical mediator of transformation induced by the oncogenic fibroblast growth factor receptor 3. Oncogene 2003, 22 (44), 6909-18.
(4) Burks, J.; Agazie, Y. M. Modulation of alpha-catenin Tyr phosphorylation by SHP2 positively effects cell transformation induced by the constitutively active FGFR3. Oncogene 2006, 25 (54), 7166-79.
(5) Li. J.; Reed, S. A.; Johnson, S. E. Hepatocyte growth factor (HGF) signals through SHP2 to regulate primary mouse myoblast proliferation. Experimental cell research 2009, 315 (13), 2284-92
(6) Miura, K.; Wakayama, Y., Tanino, M., Orba, Y., Sawa, H., Hatakeyama, M.; Tanaka, S.; Sabe, H.; Mochizuki, N. Involvement of EphA2-mediated tyrosine phosphorylation of Shp2 in Shp2-regulated activation of extracellular signal-regulated kinase. Oncogene 2013, 32 (45), 5292-5301.
(7) Matalkah, F.; Martin, E.; Zhao, H.; Agazie, Y. M. SHP2 acts both upstream and downstream of multiple receptor tyrosine kinases to promote basal-like and triple-negative breast cancer. Breast cancer research 2016, 18 (1), 2.
(8) Zhou, X.; Coad, J.; Ducatman, B.; Agazie, Y. M. SHP2 is up-regulated in breast cancer cells and in infiltrating ductal carcinoma of the breast, implying its involvement in breast oncogenesis. Histopathology 2008, 53 (4), 389-402.
(9) Zhao, H.; Agazie, Y. M. Inhibition of SHP2 in basal-like and triple-negative breast cells induces basal-to-luminal transition, hormone dependency, and sensitivity to anti-hormone treatment. BMC cancer 2015, 15, 109.
(10) Zhao, H.: Martin, E.; Matalkah, F.; Shah, N.; Ivanov, A.; Ruppert, J. M.; Lockman, P. R., Agazie, Y. M. Conditional knockout of SHP2 in ErbB2 transgenic mice or inhibition in HER2-amplified breast cancer cell lines blocks oncogene expression and tumorigenesis. Oncogene 2019, 38 (13), 2275-2290.
(11) Zhou, X. D.; Agazie, Y. M. Inhibition of SHP2 leads to mesenchymal to epithelial transition in breast cancer cells. Cell death and differentiation 2008, 15 (6), 988-96.
(12) Feng, G. S.; Hui, C. C.; Pawson, T. SH2-containing phosphotyrosine phosphatase as a target of protein-tyrosine kinases. Science 1993, 259 (5101), 1607-11.
(13) Feng, G. S.; Shen, R.; Heng, H. H.; Tsui, L. C.; Kazlauskas, A.; Pawson, T. Receptor-binding, tyrosine phosphorylation and chromo-some localization of the mouse SH2-containing phosphotyrosine phosphatase Syp. Oncogene 1994, 9 (6), 1545-50.
(14) Hof, P.; Pluskey, S.; Dhe-Paganon, S.; Eck, M. J.; Shoelson, S. E. Crystal structure of the tyrosine phosphatase SHP-2. Cell 1998, 92 (4), 441-50.
(15) Zhou, X. D.; Agazie, Y. M. Molecular Mechanism for SHP2 in Promoting HER2-induced Signaling and Transformation. J. Biol. Chem. 2009, 284 (18), 12226-12234.
(16) Hartman, Z. R.; Schaller, M. D.; Agazie, Y. M. The tyrosine phosphatase SHP2 regulates focal adhesion kinase to promote EGF-induced lamellipodia persistence and cell migration. Molecular cancer research: MCR 2013, 11 (6), 651-64.
(17) Zhang, S. Q.; Yang, W.; Kontaridis, M. I.; Bivona, T. G.; Wen, G.; Araki, T.; Luo, J.; Thompson, J. A.; Schraven, B. L.; Philips, M. R.; Neel, B. G. Shp2 regulates SRC family kinase activity and Ras/Erk activation by controlling Csk recruitment. Molecular cell 2004, 13 (3), 341-55.
(18) Hellmuth, K.; Grosskopf, S.; Lum, C. T.; Wurtele, M.; Roder, N., von Kries, J. P.; Rosario, M.; Rademann, J.; Birchmeier, W. Specific inhibitors of the protein tyrosine phosphatase Shp2 identified by high-throughput docking. Proc. Natl. Acad. Sci. U.S.A. 2008, 105(20), 7275-80.
(19) Chen, L.; Pernazza, D.; Scott, L. M.; Lawrence, H. R.; Ren, Y.; Luo, Y.; Wu, X.; Sung, S. S.; Guida, W. C.; Sebti. S. M.; Lawrence, N. J.; Wu, J. Inhibition of cellular Shp2 activity by a methyl ester analog of SPI-112. Biochemical pharmacology 2010, 80 (6), 801-10.
(20) Zhang, X.; He, Y.; Liu, S.; Yu, Z.; Jiang, Z. X.; Yang, Z.; Dong, Y.; Nabinger, S. C.; Wu, L.; Gunawan, A. M.; Wang, L.; Chan, R. J.; Zhang, Z. Y. Salicylic acid based small molecule inhibitor for the oncogenic Src homology-2 domain containing protein tyrosine phosphatase-2 (SHP2). Journal of medicinal chemistry 2010, 53 (6), 2482-93.
(21) Zeng, L. F.; Zhang, R. Y.; Yu, Z. H.; Li, S.; Wu, L.; Gunawan, A. M.; Lane, B. S.; Mali, R. S.; Li, X.; Chan, R. J.; Kapur, R.; Wells, C. D.; Zhang, Z. Y. Therapeutic potential of targeting the oncogenic SHP2 phosphatase. Journal of medicinal chemistry 2014, 57 (15), 6594-609.
(22) Yu, B.; Liu, W.; Yu, W. M.; Loh, M. L.; Alter, S.; Guvench, O.; Mackerell, A. D.; Tang, L. D.; Qu, C. K. Targeting Protein Tyrosine Phosphatase SHP2 for the Treatment of PTPN11-Associated Malignancies. Molecular cancer therapeutics 2013, 12 (9), 1738-1748.
(23) Mostinski, Y.; Heynen, G. J. J. E.; Lopez-Alberca, M. P.; Paul, J.; Miksche, S.; Radetzki, S.; Schaller, D.; Shanina, E.; Seyffarth, C.; Kolomeets, Y.; Ziebart, N.; de Schryver, J.; Oestreich, S.; Neuenschwander, M.; Roske, Y.; Heinemann, U.; Rademacher, C.; Volkamer, A.; von Kries, J. P.; Birchmeier, W.; Nazare, M. From Pyrazolones to Azaindoles: Evolution of Active-Site SHP2 Inhibitors Based on Scaffold Hopping and Bioisosteric Replacement. Journal of medicinal chemistry 2020, 63 (23), 14780-14804.
(24) Hartman, Z.; Geldenhuys, W. J.; Agazie, Y. M. Novel Small-Molecule Inhibitor for the Oncogenic Tyrosine Phosphatase SHP2 with Anti-Breast Cancer Cell Effects. Acs Omega 2020, 5 (39), 25113-25124.
(25) Chen, Y. N.; LaMarche, M. J.; Chan, H. M.; Fekkes, P.; Garcia-Fortanet, J.; Acker, M. G.; Antonakos, B.; Chen, C. H.; Chen, Z.; Cooke, V. G.; Dobson, J. R.; Deng, Z.; Fei, F.; Firestone, B.; Fodor, M.; Fridrich, C.; Gao, H.; Grunenfelder, D.; Hao, H. X.; Jacob, J.; Ho, S.; Hsiao, K.; Kang, Z. B.; Karki, R.; Kato, M.; Larrow, J.; La Bonte, L. R.; Lenoir, F.; Liu, G.; Liu, S.; Majumdar, D.; Meyer, M. J.; Palermo, M.; Perez, L.; Pu, M.; Price, E.; Quinn, C.; Shakya, S.; Shultz, M. D.; Slisz, J.; Venkatesan, K.; Wang, P.; Warmuth, M.; Williams, S.; Yang, G.; Yuan, J.; Zhang, J. H.; Zhu, P.; Ramsey, T.; Keen, N. J.; Sellers, W. R.; Stams, T.; Fortin, P. D. Allosteric inhibition of SHP2 phosphatase inhibits cancers driven by receptor tyrosine kinases. Nature 2016, 535 (7610), 148-52.
(26) Garcia Fortanet, J.; Chen, C. H.; Chen, Y. N.; Chen, Z.; Deng, Z.; Firestone, B.; Fekkes, P.; Fodor, M.; Fortin, P. D.; Fridrich, C.; Grunenfelder, D.; Ho, S.; Kang, Z. B.; Karki, R.; Kato, M.; Keen, N.; LaBonte, L. R.; Larrow, J.; Lenoir, F.; Liu, G.; Liu, S.; Lombardo, F.; Majumdar, D.; Meyer, M. J.; Palermo, M.; Perez, L.; Pu, M.; Ramsey, T.; Sellers, W. R.; Shultz, M. D.; Stams, T.; Towler, C.; Wang, P.; Williams, S. L.; Zhang, J. H.; LaMarche, M. J. Allosteric Inhibition of SHP2: Identification of a Potent, Selective, and Orally Efficacious Phosphatase Inhibitor. Journal of medicinal chemistry 2016, 59 (17), 7773-82.
(27) Czako, B.; Sun, Y. T.; McAfoos, T.; Cross, J. B.; Leonard, P. G.; Burke, J. P.; Carroll, C. L.; Feng, N. P.; Harris, A. L.; Jiang, Y. Y.; Kang, Z. J.; Kovacs, J. J.; Mandal, P.; Meyers, B. A.; Msech, F.; Parker, C. A.; Yu, S. S.; Williams, C. C.; Wu, Q.; Di Francesco, M. E.; Draetta, G.; Heffernan, T.; Marszalek, J. R.; Kohl, N. E.; Jones, P. Discovery of 6-[(3S,4S)-4-Amino-3-methyl-2-oxa-8-azaspiro[4.5]decan-8-yl]-3-(2,3-dichlorophenyl)-2-methyl-3,4-dihydropyrimidin-4-one (IACS-15414), a Potent and Orally Bioavailable SHP2 Inhibitor. Journal of medicinal chemistry 2021, 64 (20), 15141-15169.
(28) Halgren, T. A.; Murphy, R. B.; Friesner, R. A.; Beard, H. S.; Frye, L. L.; Pollard, W. T.; Banks, J. L. Glide: A new approach for rapid, accurate docking and scoring. 2. Enrichment factors in database screening. Journal of medicinal chemistry 2004, 47 (7), 1750-1759.
(29) Yang, J.; Liu, L.; He, D.; Song, X.; Liang, X.; Zhao, Z. J.: Zhou, G. W. Crystal structure of human protein-tyrosine phosphatase SHP-1. J. Biol. Chem. 2003, 278 (8), 6516-20.
(30) Krishnan, N.; Bencze, G.; Cohen, P.; Tonks, N. K. The anti-inflammatory compound BAY-11-7082 is a potent inhibitor of protein tyrosine phosphatases. FEBS journal 2013, 280 (12), 2830-41.
(31) Lopez, S. M.; Hodgson, M. C.; Packianathan, C.; Bingol-Ozakpinar, O.; Uras, F.; Rosen, B. P.; Agoulnik, I. U. Determinants of the tumor suppressor INPP4B protein and lipid phosphatase activities. Biochemical and biophysical research communications 2013, 440 (2), 277-82.
(32) Hartman, Z.; Geldenhuys, W. J.; Agazie, Y. M. A specific amino acid context in EGFR and HER2 phosphorylation sites enables selective binding to the active site of Src homology phosphatase 2 (SHP2). J. Biol. Chem. 2020, 295 (11), 3563-3575.
(33) Montalibet, J.; Skorey, K. I.; Kennedy, B. P. Protein tyrosine phosphatase: enzymatic assays. Methods 2005, 35 (1), 2-8.
(34) Al-Amin, R. A.; Gallant, C. J.; Muthelo, P. M.; Landegren, U. Sensitive Measurement of Drug-Target Engagement by a Cellular Thermal Shift Assay with Multiplex Proximity Extension Readout. Anal. Chem. 2021, 93 (31), 10999-11009.
(35) Zhou, X.; Agazie, Y. M. Molecular mechanism for SHP2 in promoting HER2-induced signaling and transformation. J. Biol. Chem. 2009, 284 (18), 12226-34.
(36) Fillmore, C. M.; Kuperwasser, C. Human breast cancer cell lines contain stem-like cells that self-renew, give rise to phenotypically diverse progeny and survive chemotherapy. Breast cancer research: BCR 2008, 10 (2), R25.
(37) LaRochelle, J. R.; Fodor, M.; Xu, X.; Durzynska, I.; Fan, L.; Status, T.; Chan, H. M.; LaMarche, M. J.; Chopra, R.; Wang, P.; Fortin, P. D.; Acker, M. G.; Blacklow, S. C. Structural and Functional Consequences of Three Cancer-Associated Mutations of the Oncogenic Phosphatase SHP2. Biochemistry 2016, 55 (15), 2269-2277.
(38) Dontu, G.; Abdallah, W. M.; Foley, J. M.; Jackson, K. W.; Clarke, M. F.; Kawamura, M. J.; Wicha, M. S. In vitro propagation and transcriptional profiling of human mammary stem/progenitor cells. Genes & development 2003, 17 (10), 1253-70.

It will be appreciated by those persons of ordinary skill in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A compound of the formula: 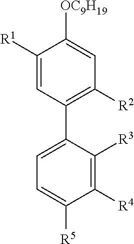
wherein R¹ is a hydrogen or a COOH, R² is a hydrogen or a COOH, R³ is a hydrogen or a COOH, R⁴ is selected from one of the group consisting of H, COOH, and CH₂COOH, and R⁵ is selected from one of the group consisting of H, COOH, and CH₂COOH.

2. The compound of claim 1 that is a phosphotyrosyl phosphatase 2 inhibitor.

3. The compound of claim 1 wherein R¹ is COOH, and wherein R², R³, and R⁴ are each H, and wherein R⁵ is CH₂COOH.

4. The compound of claim 1 wherein R¹, R³, and R⁴ are each H, and wherein R² is COOH, and wherein R⁵ is CH₂COOH.

5. A pharmaceutical composition comprising a compound having the formula: 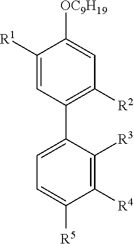
wherein R¹ is a hydrogen or a COOH, R² is a hydrogen or a COOH, R³ is a hydrogen or a COOH, R⁴ is one selected from the group consisting of H, COOH, and CH₂COOH, and R⁵ is one selected from the group consisting of H, COOH, and CH₂COOH, and
at least one acceptable pharmaceutical carrier.

6. The pharmaceutical composition of claim 5 including wherein said compound has the formula wherein R¹ is COOH, and wherein R², R³, and R⁴ are each H, and wherein R⁵ is CH₂COOH.

7. The pharmaceutical composition of claim 5 including wherein said compound has the formula wherein R¹, R³, and R⁴ are each H, and wherein R² is COOH, and wherein R⁵ is CH₂COOH.

8. A method of treating a patient having cancer comprising administering a therapeutically effective amount of a compound of the formula: 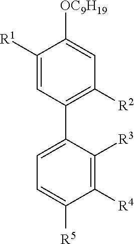
wherein R¹ is a hydrogen or a COOH, R²is a hydrogen or a COOH, R³ is a hydrogen or a COOH, R⁴ is selected from one of the group consisting of H, COOH, and CH₂COOH, and R⁵ is selected from one of the group consisting of H, COOH, and CH₂COOH, for treating said patient.

9. The method of claim 8 including wherein said compound has the formula wherein R¹ is COOH, and wherein R², R³, and R⁴ are each H, and wherein R⁵ is CH₂COOH.

10. The method of claim 8 including wherein said compound has the formula wherein R¹, R³, and R⁴ are each H, and wherein R² is COOH, and wherein R⁵ is CH₂COOH.

11. A method of treating a patient having cancer comprising administering a therapeutically effective amount of a pharmaceutical composition comprising a compound having the formula: 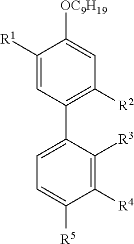
wherein R¹ is a hydrogen or a COOH, R² is a hydrogen or a COOH, R³ is a hydrogen or a COOH, R⁴ is one selected from the group consisting of H, COOH, and CH₂COOH, and R⁵ is one selected from the group consisting of H, COOH, and CH₂COOH, and
an acceptable pharmaceutical carrier, for treating said patient.

12. The method of claim 11 including wherein said compound has the formula wherein R¹ is COOH, and wherein R², R³, and R⁴ are each H, and wherein R⁵ is CH₂COOH.

13. The method of claim 11 including wherein said compound has the formula wherein R¹, R³, and R⁴ are each H, and wherein R² is COOH, and wherein R⁵ is CH₂COOH.

14. A compound that is a derivative of CNBDA that is a phosphotyrosyl phosphatase 2 inhibitor.

15. A compound of the formula 4′-(carboxymethyl)-4-(nonyloxy)-[1,1′-biphenyl]-3-carboxylic acid (BPDA2).

16. A compound having the formula: 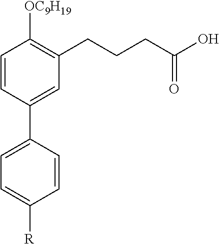
wherein R is COOH or CH₂COOH.

17. A pharmaceutical composition comprising a compound having the formula: 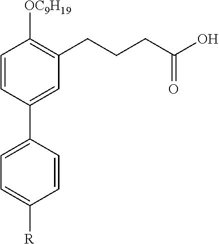
wherein R is COOH or CH₂COOH, and
at least one pharmaceutically acceptable carrier.

18. A method of treating a patient having cancer comprising administering a therapeutically effective amount of a compound of the formula: 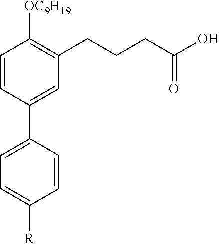
wherein R is COOH or CH₂COOH.

19. A method of treating a patient having cancer comprising administering a therapeutically effective amount of a pharmaceutical composition comprising a compound having the formula: 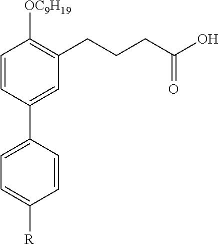
wherein R is COOH or CH₂COOH, and
at least one pharmaceutically acceptable carrier, for treating said patient.
